SEQUENCE LISTING (11 sequences)

SEQ ID NO: 1 - protein, 12 aa, Artificial Sequence
  Synthetic Peptide
RHFQTHTVPLSL

SEQ ID NO: 2 - protein, 12 aa, Artificial Sequence
  Synthetic Peptide
AHFQTHTVPLSL

SEQ ID NO: 3 - protein, 12 aa, Artificial Sequence
  Synthetic Peptide
LGTKALSYSISI

SEQ ID NO: 4 - protein, 11 aa, Artificial Sequence
  Synthetic Peptide
XXXXXXXXPXX

SEQ ID NO: 5 - protein, 12 aa, Artificial Sequence
  Synthetic Peptide
SREPQYGGPAAD

SEQ ID NO: 6 - protein, 530 aa, Norwalk Virus
MMMASKDATSSVDGASGAGQLVPEVNASDPLAMDPVAGSSTAVATAGQVNPIDPWIINNFVQAPQGEFTISPNNTPGDVLFDLSLGPHLNPFLLHLSQMYNGWVGNMRVRIMLAGNAFTAGKIIVSCIPPGFGSHNLTIAQATLFPHVIADVRTLDPIEVPLEDVRNVLFHNNDRNQQTMRLVCMLYTPLRTGGGTGDSFVVAGRVMTCPSPDFNFLFLVPPTVEQKTRPFTLPNLPLSSLSNSRAPLPISSMGISPDNVQSVQFQNGRCTLDGRLVGTTPVSLSHVAKIRGTSNGTVINLTELDGTPFHPFEGPAPIGFPDLGGCDWHINMTQFGHSSQTQYDVDTTPDTFVPHLGSIQANGIGSGNYVGVLSWISPPSHPSGSQVDLWKIPNYGSSITEATHLAPSVYPPGFGEVLVFFMSKMPGPGAYNLPCLLPQEYISHLASEQAPTVGEAALLHYVDPDTGRNLGEFKAYPDGFLTCVPNGASSGPQQLPINGVFVFVSWVSRFYQLKPVGTASSARGRLGLRR

SEQ ID NO: 7 - protein, 212 aa, Norwalk Virus
MAQAIIGAIAASTAGSALGAGIQVGGEAALQSQRYQQNLQLQENSFKHDREMIGYQVEASNQLLAKNLATRYSLLRAGGLTSADAARSVAGAPVTRIVDWNGVRVSAPESSATTLRSGGFMSVPIPFASKQKQVQSSGISNPNYSPSSISRTTSWVESQNSSRFGNLSPYHAEALNTVWLTPPGSTASSTLSSVPRGYFNTDRLPLFANNRR

SEQ ID NO: 8 - protein, 12 aa, Artificial Sequence
  Synthetic Peptide
SVSVGMKPSPRP

SEQ ID NO: 9 - protein, 12 aa, Artificial Sequence
  Synthetic Peptide
AQHTLKKWPLLV

SEQ ID NO: 10 - protein, 12 aa, Artificial Sequence
  Synthetic Peptide
QLPPNPATPRSL

SEQ ID NO: 11 - protein, 12 aa, Artificial Sequence
  Synthetic Peptide
KPTLQELTPTTL

(19) United States
(12) Patent Application Publication
(10) Pub. No.: US 2011/0020786 A1
(43) Pub. Date: Jan. 27, 2011

(54) PEPTIDE DENDRIMERS: AFFINITY REAGENTS FOR BINDING NOROVIRUSES

(75) Inventors: Timothy Palzkill, Houston, TX (US); Zanna Beharry, Houston, TX (US)

Correspondence Address: FULBRIGHT & JAWORSKI, LLP, 1301 MCKINNEY, SUITE 5100, HOUSTON, TX, 77010-3095, US

(73) Assignee: BAYLOR COLLEGE OF MEDICINE, Houston, TX (US)

(21) Appl. No.: 12/681,835

(22) PCT Filed: Oct. 7, 2008
(86) PCT No.: PCT/US2008/079068

§ 371 Date: Oct. 7, 2010

Related U.S. Application Data
(60) Provisional application No. 60/978,204, filed on Oct. 8, 2007.

(51) Int. Cl. C12Q 1/70; C07K 2/00; C12M 1/34

(52) U.S. Cl. 435/5; 530/350; 435/287.1

(57) ABSTRACT

Noroviruses are recognized as the most common cause of outbreaks of acute gastroenteritis in humans. Therefore, the present invention relates to peptides or dendrimers that bind Noroviruses and the methods for identifying and synthesizing these peptides. It also relates to the detection of Noroviruses using said peptides or dendrimers formed by them.

### RELATED APPLICATIONS

This application claims priority to U.S. Provisional Patent Application 60/978,204 filed Oct. 8, 2007, which is incorporated herein in its entirety.

### STATEMENT REGARDING FEDERALLY SPONSORED RESEARCH OR DEVELOPMENT

This work was supported by National Institutes of Health grants P01 AI057788 and 1 T32 AI55413-01. The United States has certain rights in the invention.

### TECHNICAL FIELD

The present invention at least relates generally to the fields of virology, cell biology, and molecular biology. In particular cases, it relates to Noroviruses and identifying and synthesizing peptides or dendrimers that bind Noroviruses. It also relates to the detection of Noroviruses using these peptides or dendrimers formed by them.

### BACKGROUND OF THE INVENTION

Noroviruses (NoVs) are now recognized as the most common cause of outbreaks of acute gastroenteritis in humans (Hutson, 2004). Hundreds of NoV strains have been identified throughout the world and have been classified into five groups (genogroups I-V), with the majority of human NoVs belonging to genogroups I and II, based on sequence similarity within highly conserved regions of the genome including the capsid protein and RNA polymerase (Ando, 2000). The genogroup II NoVs are the predominant causes of viral gastroenteritis outbreaks globally (Bull, 2006). NoV outbreaks occur in various settings including nursing homes, hospitals, schools, restaurants, and cruise ships, with transmission occurring through contaminated food or water or through close contact with infected persons. Underreporting of NoV outbreaks remains a problem because of the following: (1) many people recover within 2-3 days, and therefore may not report their illness; (2) there is no routine, commercially available NoV test; and (3) the capacity to test for NoV exists mainly in state and some local reference labs and is lacking in clinical labs (Blanton, 2006; Radford, 2004). Therefore, there is a need for a rapid and broadly reactive diagnostic assay for detection of NoVs.

Several methods to detect NoVs have been described in the art. U.S. Pat. No. 7,205,112 B2 discloses a method of detecting NoV RNA through RNA amplification. Antibodies i.e. monoclonal antibodies, specific to NoVs have been described previously, see for example, Yoda, 2003 or Batten, 2006. Methods of detecting NoVs using such antibodies are described in the art as well (Japanese Pat. Publication JP 2005/082558A). U.S. Patent Application No. 2005/0152911 also describes the use of monoclonal antibodies to bind to a NoV, and, in addition, teaches peptides which inhibit the antibody binding to the NoV, or inhibit NoV binding to a cell.

NoV permissive culture and culturing methods are provided in U.S. Patent Application No. 2006/003957 along with methods to detect NoVs in a biological sample using the permissive culture. Further methods to detect are also disclosed, including cytopathic assay, antibody assay, nucleic acid detection assay or protein detection

While immunoassays offer several advantages, the time and cost required for antibody production and lack of cross-reactivity for detection of various strains of NoVs demonstrates a need for new diagnostic tools. Thus, this invention is the first to demonstrate the development of NoV peptide affinity reagents for the capture and detection of virus particles.

### SUMMARY OF THE INVENTION

The present invention is directed to compositions, methods, and kits relating to peptides and/or dendrimers that bind to a Norovirus (NoV) or a Norovirus-like particle (NoVLP). For the sake of brevity, herein, when referring to peptides that bind to a NoV, or NoVLP, it should be understood that this also applies to dendrimers that bind to a NoV or NoVLP.

One embodiment of the invention relates to a composition comprising a peptide wherein the peptide binds to a Norovirus or a Norovirus-like particle. In certain aspects, the peptide may or may not be provided on a solid support structure. The peptide can also bind to a Norovirus, wherein the Norovirus is a Norwalk virus, for example. Multiple copies of the peptide can be provided on the solid support structure, for example, but not limited to, between 2 and 50 copies of the peptide.

The peptide of the current invention can be 3-50 amino acids long or 3-20 amino acids long or, more specifically, 5-18 amino acids long, 7-18 amino acids long, 8-17 amino acids long, 9-16 amino acids long, 10-15 amino acids long, 11-14 amino acids long, 10-20 amino acids long. In a particular embodiment, the peptide is 10 amino acids long, 11 amino acids long, 12 amino acids long, 13 amino acids long, 14 amino acids long, 15 amino acids long, 16 amino acids long, 17 amino acids long, 18 amino acids long, 19 amino acids long, or 20 amino acids long, for example.

In cases wherein the peptide is provided or utilized on a solid support structure, the solid support structure can be any suitable material or composition. In particular embodiments, the solid support structure is selected from the group consisting of a membrane, a filter, a chip, a slide, a wafer, a fiber, a magnetic or nonmagnetic bead, a gel, tubing, a strip, a plate, a rod, a polymer, a particle, a microparticle, a capillary, a column, a resin, a protein, and a combination thereof. It is contemplated that a resin solid support structure could comprise a Fmoc MAP resin, or more specifically, a Fmoc-8-branch MAP resin. It is also contemplated that any suitable, large protein could comprise a solid support structure, for example bovine serum albumin (BSA). In another embodiment, nonmagnetic beads could comprise polystyrene or polyacrylic beads. Any large, readily available protein could be used as the solid support. BSA was chosen as an example only because it is used in the field and is inexpensive to obtain. In some embodiments, polystrene beads used as peptide synthesis resin could perform peptide synthesis and not cleave off peptides in final step. These peptide-covered beads are used for solid support, in specific examples. In another embodiment, peptides could be attached to polyacrylic beads.

In some embodiments, the peptide is comprised in a structure can form a dendrimer. In specific cases, the dendrimer is synthesized on a solid support similar to conventional peptide synthesis and then cleaved off the bead. After cleavage from the bead, it is not on a solid support, in particular cases. In certain cases, the dendrimer is left on the bead and used with solid support, although it is standard in the art to cleave it off.

Particularly, the dendrimer can be a monovalent or multivalent dendrimer. For example, but not limited to, the multivalent dendrimers can be divalent, trivalent, tetravalent, pentavalent, hexavalent, heptavalent, octovalent, nanovalent or decavalent. Further, a multivalent dendrimer may have more branches than a decavalent dendrimer. In certain embodiments, the dendrimer may have 11-30 branches, or 11-20 branches, preferably the dendrimer would have 8, 12 or 16 branches.

In a particular embodiment of the invention, the c-terminus of the peptide comprises a sequence of a proline followed by any other two amino acids. It is contemplated that the peptide may be less than 50 amino acids long, less than 45 amino acids long, less than 40 amino acids long, less than 35 amino acids long, less than 30 amino acids long, less than 25 amino acids long, less than 20 amino acids long, or less than 15 amino acids long, for example, 12 amino acids long.

In a more specific embodiment, the peptide comprises SEQ ID NO: 4. In another embodiment, the peptide may comprise SEQ ID NO: 1 or SEQ ID NO: 2.

In specific embodiments, the Norovirus to which the peptide binds is contemplated to be selected from the group consisting of genogroup III, genogroup IV, genogroup V and a combination thereof. Specifically, the peptide may bind to genogroup I or genogroup II. In particular embodiments, the peptide binds to Norwalk virus (comprised in genogroup I). In other embodiments, the peptide binds to a genogroup II strain selected from the group consisting of Houston, Snow Mountain, Grimsby and a combination thereof.

The peptide is further contemplated to bind to the P domain (SEQ ID NO:7) of the Norovirus or Norovirus like particle.

In another embodiment, the peptide could be used in a method of detecting a Norovirus or a Norovirus-like particle in a sample. The sample may be taken from an individual or an environmental source. In a specific embodiment, the source is a water source, a sample source, or a swab of a surface, for example. The method may comprise the steps of exposing the sample to the peptide which may or may not be provided on a support structure, wherein the peptide binds to a Norovirus or a Norovirus-like particle; and detecting binding of the Norovirus or a Norovirus-like particle to the peptide, wherein binding indicates the presence of a Norovirus or a Norovirus-like particle in the sample. In certain embodiments, the detecting step comprises obtaining the Norovirus or Norovirus-like particle in a complex with the peptide to produce a Norovirus or Norovirus-like particle and peptide complex; and providing an antibody that immunologically reacts with the complex, wherein when said antibody immunologically reacts with the complex said Norovirus or Norovirus-like particle is detected. In particular embodiments, the providing step comprises detection by a secondary antibody or detection by a tertiary antibody. In further embodiments, detection may comprise the detection of a label, wherein the label is selected from a group consisting of a fluorescent label, a colorometric label, and a radioactive label. In an alternative embodiment, binding could be detected by surface plasmon resonance. In specific embodiments of the invention, the peptide is SEQ ID NO: 1, SEQ ID NO:2, SEQ ID NO: 8, SEQ ID NO: 10, SEQ ID NO: 11, and/or SEQ ID NO: 3.

In an embodiment of the invention, a peptide is used in a method of detecting a Norovirus or a Norovirus-like particle in a sample. In certain embodiments, the peptide is one or more of the peptides selected from the group consisting of SEQ ID NO: 1, SEQ ID NO: 2, SEQ ID NO: 8, SEQ ID NO: 10, SEQ ID NO: 11, and SEQ ID NO: 3. In a specific embodiment of the invention, SEQ ID NO:8 is used to detect the Grimsby virus. In another specific embodiment, the peptide has 70% identity to, 80% identity to, 85% identity to, 90% identity to, 95% identity to, or 100% identity to SEQ ID NO: 1, SEQ ID NO:2, SEQ ID NO: 8, SEQ ID NO: 10, SEQ ID NO: 11, or SEQ ID NO: 3. In another embodiment of the invention, a proline occupies the fourth to the last amino acid position in the peptide and/or a leucine occupies the last position of the peptide.

Another embodiment of the invention comprises a method of treating and/or preventing a Norovirus infection in an individual comprising the step of delivering to the individual an effective amount of a peptide that binds to a Norovirus or a Norovirus-like particle. The delivered peptide may or may not be provided in a dendrimeric branched structure.

Yet another embodiment comprises a method of identifying peptides that bind to a Norovirus or a Norovirus-like particle comprising the steps of expressing the capsid protein of a Norovirus in a cell to form noninfectious virus like particles identical to Norovirus-like particles with the same structural organization as Norovirus-like particles but lacking genetic material; exposing a phage display peptide library to the noninfectious virus-like particles; and identifying peptides that bind to the noninfectious virus-like particles. In certain aspects, the method further comprises providing the identified peptides on a solid support structure. In one embodiment the caspid protein is expressed in an insect cell.

In particular embodiments, VLPs are made by expressing ORF2 and (optionally) ORF3 capsid proteins in insect cells. The ORF2 capsid protein self-assembles into a round particle that resembles the virus, only there is no RNA and so it is not infectious. Without RNA there is no way for viral replication to occur. It is also lacking other viral proteins, for example.

A further embodiment comprises a kit for detecting, treating and/or preventing a Norovirus or a Norovirus-like particle infection in an individual comprising a peptide provided that binds to a Norovirus or a Norovirus-like particle. In one embodiment, the peptide may or may not be provided in a dendrimeric branched structure.

A further embodiment regards a composition comprising a polynucleotide encoding a peptide, wherein the polynucleotide is selected from the group consisting of (a) a polynucleotide encoding an amino acid sequence selected from the group consisting of SEQ ID NO: 1 and SEQ ID NO: 2; (b) a polynucleotide encoding an amino acid sequence having at least 80%, at least 85%, at least 90%, at least 95%, at lease 97%, or at least 99% identity with the polynucleotide sequence of (a); (c) a polynucleotide that hybridizes with the polynucleotide of (a) under hybridization conditions of 0.02 M to about 0.15 M NaCl at temperatures of about 50° C. to about 70° C.; and (d) a polynucleotide that is complementary to the polynucleotide of (a), (b), or (c).

Another embodiment comprises an expression vector comprising a polynucleotide encoding a peptide, wherein the polynucleotide is selected from the group consisting of (a) a polynucleotide encoding an amino acid sequence selected from the group consisting of SEQ ID NO: 1 and SEQ ID NO: 2; (b) a polynucleotide encoding an amino acid sequence having at least 80%, at least 85%, at least 90%, at least 95%, at lease 97%, or at least 99% identity with the polynucleotide of (a); (c) a polynucleotide that hybridizes with the polynucleotide of (a) under hybridization conditions of 0.02 M to about 0.15 M NaCl at temperatures of about 50° C. to about 70° C.; and (d) a polynucleotide that is complementary to the polynucleotide of (a), (b), or (c).

The expression vector may be any vector suitable to comprise a polynucleotide. In specific embodiments, the expression vector may comprise a viral vector or a plasmid vector. In certain aspects, a viral vector may be an adenoviral vector, an adeno-associated viral vector, a retroviral vector, a lentiviral vector, a herpes viral vector, polyoma viral vector or hepatitis B viral vector. The expression vector may also be comprised in a non-viral delivery system and such a non-viral delivery system may comprise one or more lipids.

A specific embodiment further comprises a method of treating and/or preventing a Norovirus infection in an individual comprising the step of delivering to an individual an effective amount of a polynucleotide encoding a peptide, wherein the polynucleotide is selected from the group consisting of (a) a polynucleotide encoding an amino acid sequence selected from the group consisting of SEQ ID NO: 1 and SEQ ID NO: 2, SEQ ID NO: 8, SEQ ID NO: 10, SEQ ID NO: 11, and SEQ ID NO: 3; (b) a polynucleotide encoding an amino acid sequence having at least 80%, at least 85%, at least 90%, at least 95%, at lease 97%, or at least 99% identity with the polynucleotide sequence of (a); (c) a polynucleotide that hybridizes with the polynucleotide of (a) under hybridization conditions of 0.02 M to about 0.15 M NaCl at temperatures of about 50° C. to about 70° C.; and (d) a polynucleotide that is complementary to the polynucleotide of (a), (b), or (c). In another embodiment of the invention, the polypeptide is attached to another protein.

Another specific embodiment comprises a method of treating and/or preventing a Norovirus infection in an individual comprising the step of delivering to an individual an effective amount of an expression vector comprising a polynucleotide encoding a peptide, wherein the polynucleotide is selected from the group consisting of (a) a polynucleotide encoding an amino acid sequence selected from the group consisting of SEQ ID NO: 1 and SEQ ID NO: 2, SEQ ID NO: 8, SEQ ID NO: 10, SEQ ID NO: 11, and SEQ ID NO: 3; (b) a polynucleotide encoding an amino acid sequence having at least 80%, at least 85%, at least 90%, at least 95%, at lease 97%, or at least 99% identity with the polynucleotide sequence of (a); (c) a polynucleotide that hybridizes with the polynucleotide of (a) under hybridization conditions of 0.02 M to about 0.15 M NaCl at temperatures of about 50° C. to about 70° C.; and (d) a polynucleotide that is complementary to the polynucleotide of (a), (b), or (c).

The foregoing has outlined rather broadly the features and technical advantages of the present invention in order that the detailed description of the invention that follows may be better understood. Additional features and advantages of the invention will be described hereinafter which form the individual of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1B illustrate phage display peptides and binding of the peptides to NoVLPs. FIG. 1A shows a comparison of phage ELISA binding to NoVLPs (black bars in order from left to right represent Norwalk, Houston, Snow Mountain, Grimsby) and nonspecific binding (white bars in order from left to right represent alcohol dehydrogenase, β-amylase, carbonic anhydrase, and green fluorescent protein). The control was M13 phage displaying no peptide. FIG. 1B lists corresponding sequences of phage displayed peptides illustrated in FIG. 1A. Clones 3, 4, and 6 that showed specificity for binding to NoVLPs showed some sequence identity at amino acid positions 9 and 12 (underlined).

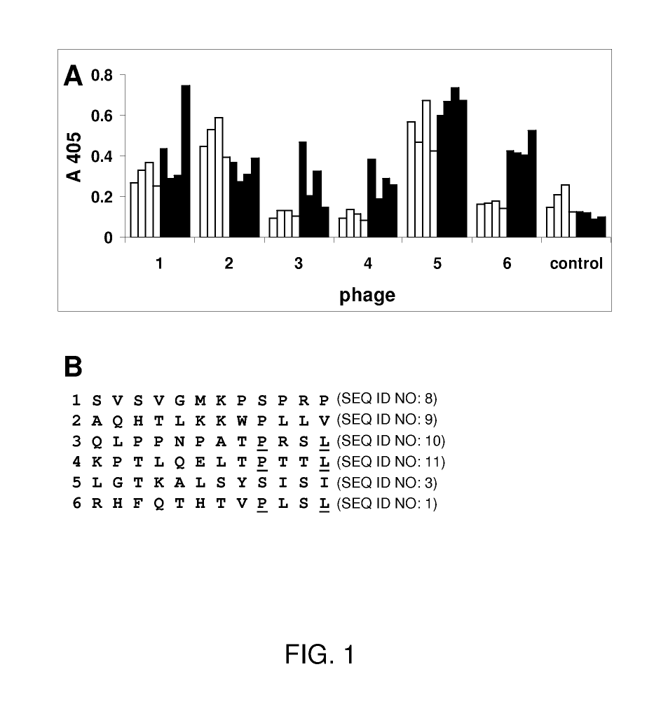

FIG. 2 shows a comparative analysis of binding of the linear RHFQTHTVPLSL peptide (SEQ ID NO: 1) (1.3 mM), RHFQTHTVPLSL (SEQ ID NO: 1) coupled to bovine serum albumin (BSA) (15 μM conjugate), and BSA (30 μM) alone to immobilized NoVLPs using surface plasmon resonance (SPR). Samples were prepared in the running buffer (HBS-P) and injected at a flowrate of 10 μL/min.

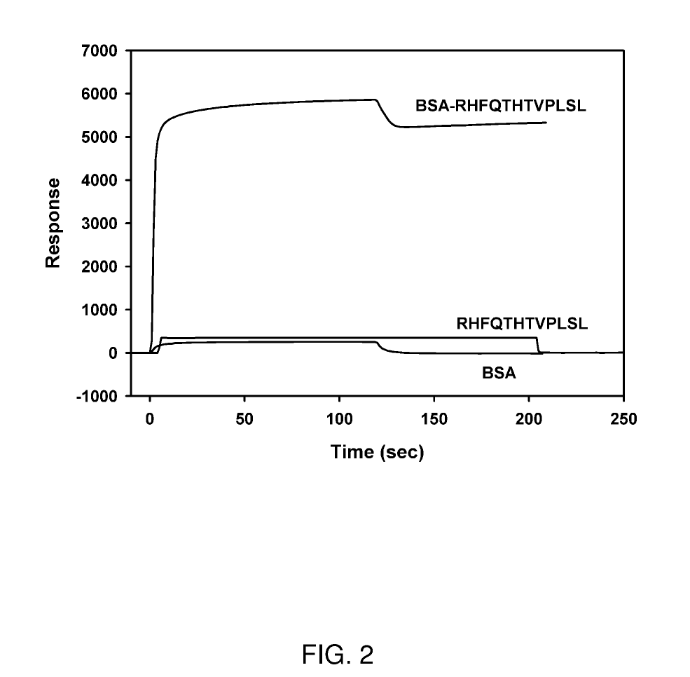

FIG. 3 is a schematic representation of the octovalent peptide dendrimer synthesized to evaluate multivalent binding.

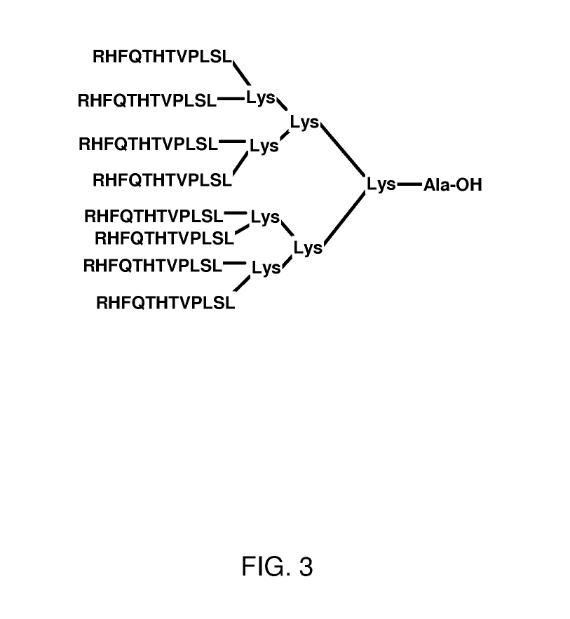

FIGS. 4A-4B are charts illustrating the concentration dependence of RHFQTHTVPLSL₈ (the octovalent dendrimer of SEQ ID NO: 1) binding to immobilized NoVLPs. 1-8 μM; 2-32 μM; 3-64 μM; 4-96 μM; 5-160 μM; 6-240 μM; (FIG. 4A). Samples were prepared in the running buffer (HBS-P) and injected at a flowrate of 10 μL/min. The arrow indicates the end of the injection of the dendrimer (binding phase) which is followed by washing with running buffer (dissociation phase). The chart in FIG. 4B shows K_{D} determination from equilibrium average response corresponding to the concentrations of RHFQTHTVPLSL₈ (the octovalent dendrimer of SEQ ID NO: 1) used for binding analysis. Data were fit using the steady-state model in the BIAevaluation software 4.1 (Biacore) resulting in a K_{D} value of 117 μM.

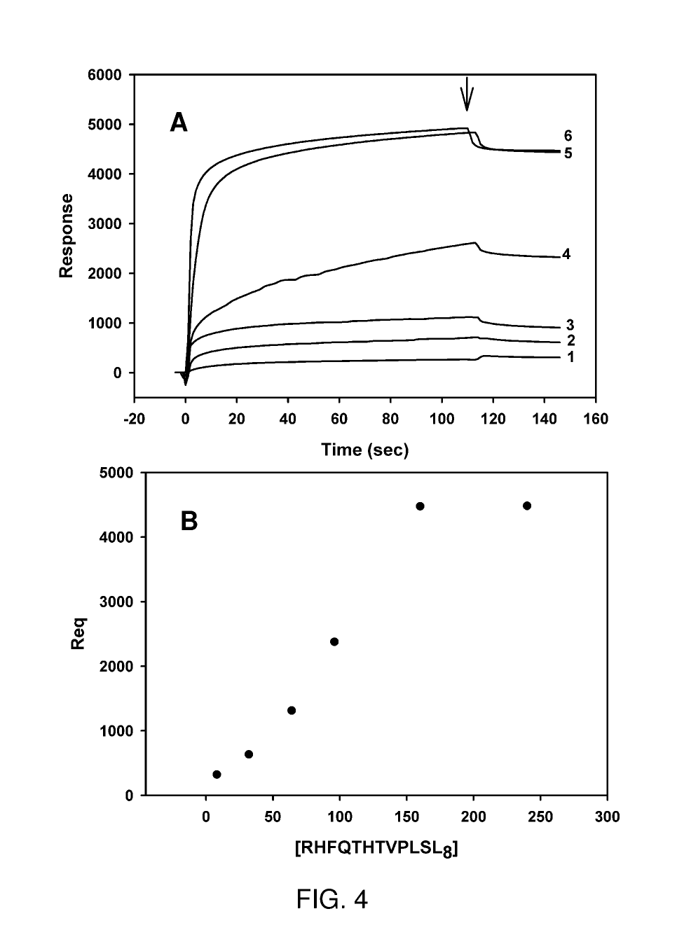

FIG. 5 shows comparative ELISA evaluations of genogroup I (Norwalk (NV)) and II (Snow Mountain (SMV), Houston (HOV), Grimsby (GRV)) virus-like particles (VLPs) binding to RHFQTHTVPLSL₈ (the octovalent dendrimer of SEQ ID NO: 1) (black bars) and the negative control SREPQYGGPAAD₈ (the octovalent dendrimer of SEQ ID NO: 5) (white bars).

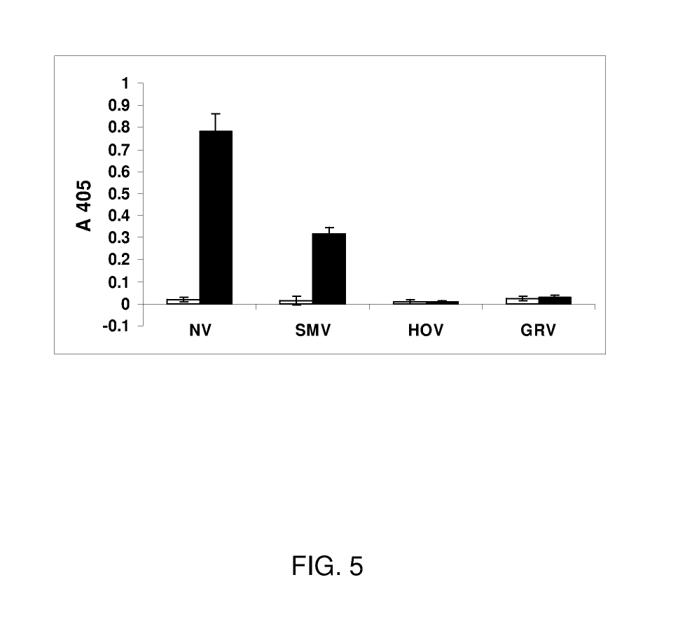

FIGS. 6A-6B show comparative evaluations of genogroup I and genogroup II binding to AHFQTHTVPLSL₈ (the octovalent dendrimer of SEQ ID NO: 2). FIG. 6A illustrates ELISA evaluations of genogroup I (NV) and II (SMV, HOV, GRV) VLPs binding to AHFQTHTVPLSL₈ (the octovalent dendrimer of SEQ ID NO: 2) (black bars) and the negative control SREPQYGGPAAD₈ (the octovalent dendrimer of SEQ ID NO: 5) (white bars). FIG. 6B depicts the analysis of AHFQTHTVPLSL₈ (the octovalent dendrimer of SEQ ID NO: 2) binding to immobilized VLPs using SPR. Samples were prepared in the running buffer (HBS-P) and injected at a flowrate of 10 μL/min. SPR traces are offset vertically for clarity. The equilibrium average response was 6748 RU (NV), 6707 RU (HOV), 6426 RU (SMV), and 6381 RU (GRV).

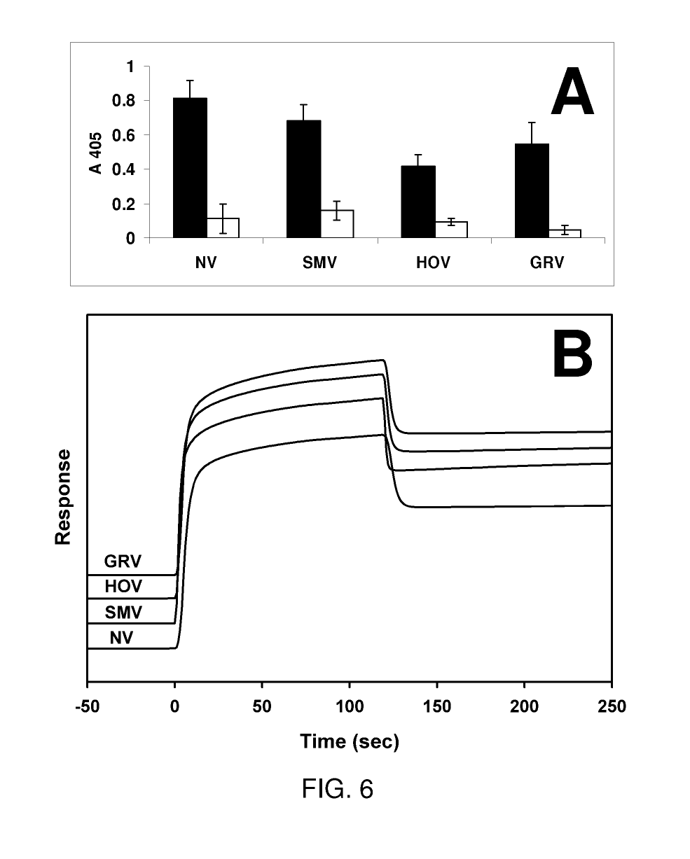

FIG. 7 illustrates comparative evaluations of binding of RHFQTHTVPLSL₈ (the octovalent dendrimer of SEQ ID NO: 1) (0.8 mg/mL, 64 μM) and AHFQTHTVPLSL₈ (the octovalent dendrimer of SEQ ID NO: 2) (0.8 mg/mL, 71 μM) to the immobilized P-domain glutathione transferase fusion protein and the negative control TEM-1 (a serine β-lactamase) and fosfomycin resistance protein (FosA, a metalloglutathione transferase). Samples were prepared in the running buffer (HBS-P) and injected at a flowrate of 10 μL/min. The arrow indicates the end of the injection of the dendrimer (binding phase) which is followed by washing with buffer (dissociation phase).

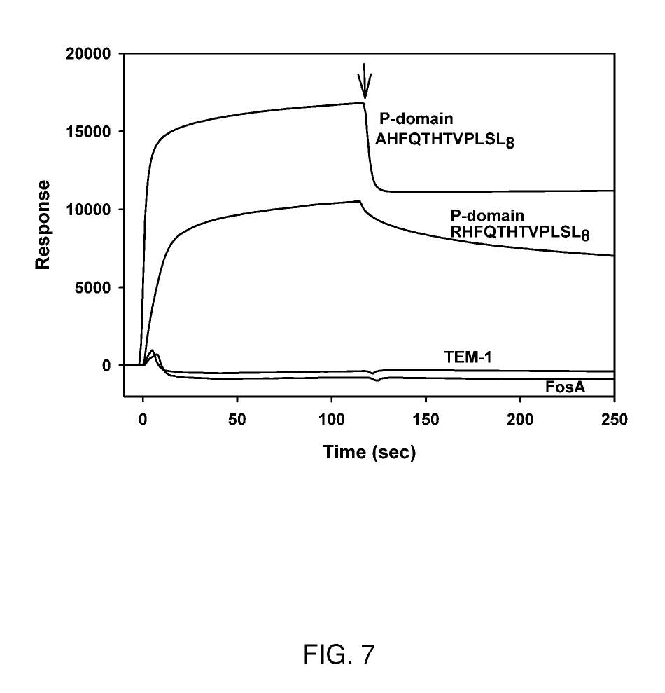

FIG. 8 shows a chart of the capture of native genogroup I (GI #1-3)) and genogroup II (GII #1-3) Norovirus from clinical samples as evaluated by ELISA. The capture reagent was RHFQTHTVPLSL₈ (the octovalent dendrimer of SEQ ID NO: 1) (middle bars), or AHFQTHTVPLSL₈ (the octovalent dendrimer of SEQ ID NO: 2) (right bars) and bound virus was detected with a MAb3912 (GI) or NS14 (GII). No genogroup II virus could be detected using RHFQTHTVPLSL₈ (the octovalent dendrimer of SEQ ID NO: 1) as the capture reagent. For comparison, detection of Norovirus from genogroup I samples (GI#1-3) was evaluated using a genogroup I specific antibody as the capture reagent (left bars). The potential of false positive signals was evaluated using clinical samples negative for Norovirus (negative #1-3) with anti-genogroup I antibody, RHFQTHTVPLSL₈ (the octovalent dendrimer of SEQ ID NO: 1) and AHFQTHTVPLSL₈ (the octovalent dendrimer of SEQ ID NO: 2) as capture reagents.

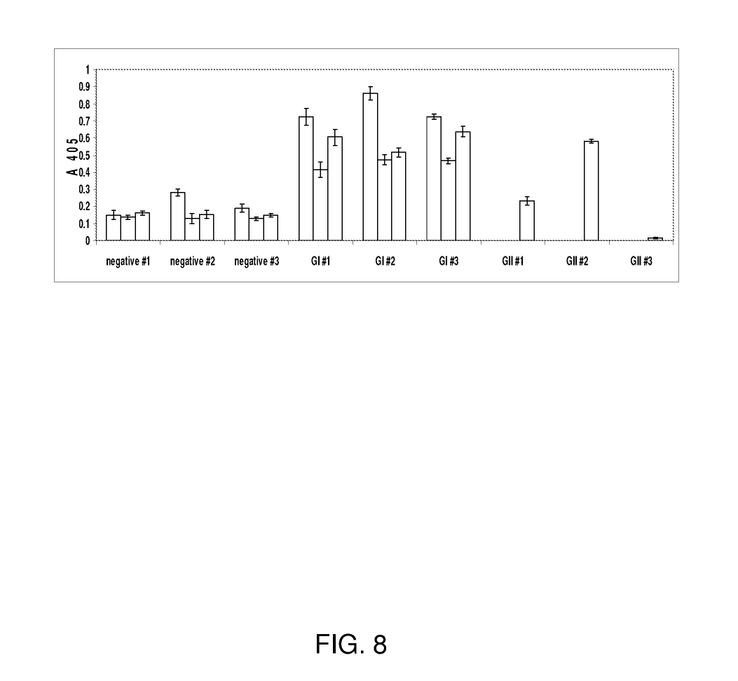

### DETAILED DESCRIPTION OF THE INVENTION

### I. Definitions

Unless defined otherwise, technical and scientific terms used herein have the same meaning as commonly understood by one of ordinary skill in the art to which this invention belongs. For purposes of the present invention, the following terms are defined below.

In keeping with long-standing patent law convention, the words “a” and “an” when used in the present specification in concert with the word comprising, including the claims, denote “one or more.” Some embodiments of the invention may consist of or consist essentially of one or more elements, method steps, and/or methods of the invention. It is contemplated that any method or composition described herein can be implemented with respect to any other method or composition described herein.

As used herein, the terms “capsid” or “capsid protein” refer to the outer protein shell of a virus. The shell or “capsid” is composed of multiple copies of the capsid protein. The genetic material of the virus is contained within the capsid.

As used herein, the term “dendrimer” refers to repeatedly branched molecules. More specifically, dendrimers refer to branched polymers with peptides attached centrally to a core matrix

As used herein, the term “effective amount” refers to an amount of the agent that will decrease or reduce the effects of or provide beneficial results relating to gastroenteritis or a Norovirus infection. Thus, an effective amount is an amount sufficient to detectably and repeatedly ameliorate, reduce, minimize or limit the extent of the disease or at least one of its symptoms.

As used herein, the terms “identity” or “similarity”, as known in the art, are relationships between two or more polypeptide sequences or two or more polynucleotide sequences, as determined by comparing the sequences. In the art, identity also means the degree of sequence relatedness between polypeptide or polynucleotide sequences, as the case may be, as determined by the match between strings of such sequences. Both identity and similarity can be readily calculated by known methods such as those described in: Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991. Methods commonly employed to determine identity or similarity between sequences include, but are not limited to those disclosed in Carillo, H., and Lipman, D., SIAM J Applied Math., 48:1073 (1988). Methods to determine identity and similarity are codified in publicly available computer programs. Preferred computer program methods to determine identity and similarity between two sequences include, but are not limited to, GCG program package, Devereux, J., et al., Nucleic Acids Research, 12(1), 387 (1984), BLASTP, BLASTN, and FASTA Atschul, S. F. et al., J Molec. Biol., 215, 403 (1990).

As used herein, an “individual” is an appropriate subject for the method of the present invention. An individual may be a mammal and in specific embodiments is any member of the higher vertebrate class Mammalia, including humans; characterized by live birth, body hair, and mammary glands in the female that secrete milk for feeding the young. Additionally, mammals are characterized by their ability to maintain a constant body temperature despite changing climatic conditions. Examples of mammals are humans, cats, dogs, cows, mice, rats, and chimpanzees. Individuals may also be referred to as “patients” or “subjects”. In one embodiment of the invention the individual is human.

As used herein, “sample” refers to a representative part of item from a larger whole. The sample may be from an individual or an environmental source. For example, a sample from an individual may comprise drawing the individual's blood or taking a sample of the individual's stool. The sample may also be taken from an environmental source. For example a sample may be taken from a water source or swabbed from a surface such as a door handle. One of skill in the art knows appropriate sample collecting and processing protocols.

The term “Noninfectious virus like particle” refers to the product of the expression of ORF2 (SEQ ID NO: 6) and in some cases ORF3 capsid (SEQ ID NO: 7) proteins in insect cells. These capsid proteins, when expressed in insect cells, self assemble into a virus like particle (VLP) lacking genetic material. The resulting VLP has the same structural organization as Norovirus like particles, without the genetic material, rendering the VLP noninfectious. Expression of the ORF2 and (optionally) ORF3 capsid proteins in insect cells results in the self assembly of the major capsid protein encoded by ORF2 into a virus like particle (VLP). The virus like particle resembles a virus but does not contain nucleic acid (RNA) and is thus not infectious. A skilled artisan recognizes that the ORF2 and ORF3 encode the capsid protein VP1 that, when expressed in insect cells, for example, self assembles into a particle that by electron microscopy (for example) looks like virus. Prasad et al. (1999) describes the structure of this VLP, for example (see for example, protein data bank structure 1IHM). Thus, in specific embodiments, “same structural organization” refers to the fact that VP1 is the capsid protein that gives the virus its form in both VLP and virus, for example. Bertolotti-Ciarlet et al. (2003) provides a reference in the art on virus like particles.

Some proteins have the intrinsic property of being able to aggregate to form larger particles. The capsid protein of the Norwalk virus has this ability. As used herein, the term “Norovirus-like particle” or “NoVLP” refers to the particle formed by the Norovirus capsid protein. The particle may or may not contain within it the genetic material of the virus. If the NoVLP does not contain any genetic material it may be referred to as a noninfectious virus like particle.

“P domain” as used herein refers to the most distal and exposed surface of the Norovirus-like particle (NoVLP). Amino acids 226-520 of SEQ ID: 6 make up the P-domain sequence. This domain can be broken down to two subdomains: P1 (amino acids 226-278 and 406-520 of SEQ ID NO: 6) and P2 (amino acids 279-405 of SEQ ID NO: 6).

The terms “prevention” and “preventing” as used herein are used according to their ordinary and plain meaning to mean “acting before” or such an act. In the context of a particular disease or health-related condition, those terms refer to delivery or application of an agent, drug, or remedy to an individual or performance of a procedure or modality on an individual for the purpose of blocking or lessening the onset of a disease or health-related condition.

The terms “protein domain” or “domain” used herein refer to a structural domain or element within a protein. Domains may have distinctly defined functions, structures, or locations, and may fold independent from the rest of the protein. Exemplary domains include but are not limited to, DNA binding, hydrophobic, hydrophilic, extracellular or intracellular domains. Particularly, the present invention is concerned with domains within the capsid protein of the Norovirus. Exemplary Norovirus capsid protein domains include but are not limited to N-terminal arm, shell (S) and protruding (P).

The terms “treatment” and “treating” refer to delivery or application of a therapeutic agent to an individual or performance of a procedure or modality on an individual for the purpose of obtaining a therapeutic benefit of a disease or health-related condition. Thus, one of skill in the art realizes that a treatment may improve the disease condition, but may not be a complete cure for the disease.

### II. Embodiments of the Invention

The present invention relates to peptides or dendrimers with the ability to bind to a Norovirus (NoV) or a Norovirus-like particle (NoVLP), as well as to methods for identification and use of NoVs and NoVLPs. In certain aspects, the invention concerns individual peptides, in others, a single peptide or multiple peptides are provided on a solid support structure. It is contemplated that the solid support structure may be selected from the group consisting of membranes, filters, chips, slides, wafers, fibers, magnetic or nonmagnetic beads, gels, tubing, strips, plates, rods, polymers, particles, microparticles, capillaries, columns, resins, proteins and a combination thereof. In one embodiment, a dendrimer comprises one or more peptides that may or may not be provided on a solid support structure. In particular aspects, the peptides or dendrimer may be monovalent or multivalent, for instance, divalent, bivalent, trivalent, tetravalent, pentavalent, hexavalent, heptavalent, octovalent, nanovalent or decavalent. In some aspects, an individual peptide may be capable of binding the NoV or NoVLP, while in other aspects, binding may be via multiple peptides or the dendrimer. It is contemplated that the peptides provided on the solid support structure may be the same, for instance homogeneous, or may be different from one another, for instance, heterogeneous. One of skill in the art recognizes suitable support structures. In another embodiment, peptides of the invention may be from 3-50 amino acids in length, for instance, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids in length. In particular embodiments, the peptide may comprise SEQ ID NO: 1, SEQ ID NO: 2, SEQ ID NO: 4, SEQ ID NO: 8, SEQ ID NO: 10, SEQ ID NO: 11, SEQ ID NO: 3, or a peptide containing Pro-Xaa-Xaa, where Xaa is any amino acid at the C-terminus.

In some aspects the individual peptide, multiple peptides, or dendrimer that binds the NoV or NoVLP may be used to detect, treat or prevent a NoV or NoVLP infection in an individual. In particular embodiments, detection may comprise exposing a sample from an to the peptide, peptides or dendrimer and detecting binding of the peptide, peptides or dendrimer to the NoV or NoVLP in the sample. In another embodiment, treatment or prevention of a NoV or NoVLP infection in an individual may comprise delivering an effective amount of the peptide, peptides or dendrimer that bind the NoV or NoVLP. It is also contemplated that the peptide, peptides or dendrimer may be provided in a kit to detect, treat or prevent a NoV or NoVLP infection in an individual.

In further embodiments, a peptide, peptides, or a dendrimer that can bind a NoV or NoVLP may be identified by expressing the capsid protein in a cell to form a noninfectious virus like particle and exposing a phage display library to the noninfectious virus like particle, then identifying peptides or dendrimers that bind to the noninfectious virus like particles.

Another embodiment relates to polynucleotides encoding the identified NoV or NoVLP binding peptides or dendrimers. In one embodiment, the polynucleotides encoding the identified peptides or dendrimers may be comprised in an expression vector. In another embodiment, the expression vector comprising the polynucleotide may be used to detect, prevent or treat a NoV or NoVLP infection in an individual.

### III. Norovirus

Norovirus (NoV) describes a genus of viruses found in the Caliciviridae family. This group of viruses may also be referred to as Norwalk-like viruses taking the name from the first identified NoV, the Norwalk virus. There exist 5 genetic groupings, or genogroups, within the NoV genus (genogroup I, genogroup II, genogroup III, genogroup IV, and genogroup V). These genogroups can be further broken into different genotypes. The majority of human NoVs belong to genogroups I and II. Genogroup II viruses, containing 19 genotypes, are the most prominent and cause the majority of gastroenteritis outbreaks worldwide. Exemplary strains of genogroup II viruses include but are not limited to Houston, Snow Mountain, Grimsby. Each strain is named after the location of outbreak from which the virus was first isolated. The Norwalk strain, named after the outbreak in Norwalk, Ohio, is a member of genogroup I. Norovirus are well defined by the International Committee on Taxonomy of Viruses, (ICTBdB Management (2006). 00.012.0.03. Norovirus. In: ICTVdB The Universal Virus Database, version 4. Buchen-Osmond, C. (Ed), Columbia University, New York, USA), incorporated herein by reference. The Norwalk virus complete genome NCBI accession number is NC 001959 and the Murine norovirus 1 NCBI accession number is NC 008311.

The NoV genome encodes three open reading frames (ORFs). The first ORF (ORF1) encodes nonstructural proteins. The second open reading frame (ORF2) is the major capsid protein of the NoV (SEQ ID NO: 6). When expressed in insect cells, this capsid protein self-assembles into noninfectious virus-like particles (VLPs) that are indistinguishable from native virions morphologically and antigenically (Bertolotti-Ciarlet, 2002). The third ORF (ORF3 provided as SEQ ID NO: 7), located on the 3′ end of the viral genome, encodes a minor structural protein. While ORF3 is not required for formation of noninfectious VLPs it is associated with VLP's when it is co-expressed with ORF2 in insect cells (Glass, 2000).

The structure of the genogroup I Norwalk strain recombinant capsid has been solved and is composed of 180 copies of the major capsid protein (Prasad, 1999). The structure of the capsid protein is arranged into 3 domains: N-terminal arm (amino acids 10-49 of SEQ ID NO: 6), shell (S) (amino acids 50-225 of SEQ ID NO: 6) and protruding (P) (amino acids 226-520 of SEQ ID NO: 6). The S domain contains the amino acids required to form the icosahedral shell of the particle, and the P domain forms the protrusions arising from the particle. The P domain is made of two subdomains; P1 (amino acids 226-278 and 406-520) and P2 (amino acids 279-405). The P2 domain is situated such that it is a large insertion between two sequences of the P1 domain. The P domain is believed to be important in receptor binding and immune recognition (Hutson, 2004; Hardy, 2005; Tan, 2005).

### IV. Identifying Norovirus Binding Peptides

In one aspect of this invention, peptides with the capacity to bind to NoVs or NoVLPs are identified. In particular aspects, peptides may bind to the capsid protein of the NoV or NoVLP. In other embodiments, the peptide may bind to the P domain of the capsid protein of the NoV or NoVLP. Further, it is envisioned that the identified peptides have the capacity to bind to one or more of the NoV genogroups (genogroups I-V).

A. Expressing Norovirus Capsid Proteins

NoVs cannot be propagated in cell culture or small animal models. However, NoV cDNA can be expressed in cell free systems, bacterial cells, insect cells and mammalian cells. In particular the capsid protein of a NoV can be expressed and results in self-assembly into empty, noninfectious virus like particles (VLPs) that are morphologically and antigenically indistinguishable from NoVs isolated from clinical samples (Bertolotti-Ciarlet, 2002). Particularly, this expression is done in insect cells to produce NoVLPs.

One particular way to achieve NoV cDNA or protein expression is through the use of baculovirus transfer vectors. To achieve this, full length NoV genes, for example the ORF2 full length gene that encodes the capsid protein of the Norwalk virus (a genogroup I NoV), are cloned into the multiple cloning sites in the transfer vector. The “multiple cloning site” refers to the segment of nucleic acids in a vector that contains multiple restriction enzyme sites. These sites can typically be used to linearize the vector through standard and well known recombinant technology. Once a vector is linearized, a new segment of nucleic acids flanked with corresponding restriction sites can be connected to the vector, which re-circularizes the vector plus the new segment. This connection is achieved through ligation. “Ligation” refers to the process of forming phosphodiester bonds between two nucleic acid fragments. Baculovirus transfer vectors known in the art include but are not limited to pAcFastBAC (Gibco-BRL), pAcAB3, pAcAB4, pAcG2T, pAcDB3 (BD Biosciences), pPolh (Sigma-Aldrich), pAcIRES, pAcSec1, pORB, pVL1392 and pVL1393 (Orbigen). Vectors can be introduced into cells or cell culture by the methods described above (see expression vectors). Once introduced into the cell and expressed, viral proteins can be collected, for instance from cell lysate or culture supernatant.

One of skill in the art would appreciate that the use of a baculovirus transfer vector would be only one of several ways commonly known in the art to express a gene product or protein. Other expression methods are described above under the sub-heading of expression vectors.

B. Peptide Interactions

Peptides with the capacity to bind to a NoV or NoVLP can be identified through peptide interactions. Peptide interactions, such as peptide binding, can occur either when a peptide is soluble or immobilized. A peptide may be immobilized by providing one or more of the peptides on a solid support structure. As used herein the term “solid support structure” refers to any rigid or semi-rigid material to which a molecule, such as a peptide, protein or nucleic acid, binds or can be attached. The support can be any porous or non-porous water insoluble material, including without limitation, membranes, filters, chips, slides, wafers, fibers, magnetic or nonmagnetic beads, gels, tubing, strips, plates, rods, polymers, particles, microparticles, capillaries, and the like. The support can have a variety of surface forms, such as wells, trenches, pins, channels and pores. Particularly, a solid support structures can comprise a glass slide, a cellulose filter membrane, a bead, a column, a resin and a protein.

For ELISA, the peptide may be coated onto the surface of the plastic of ELISA wells in a standard buffer in the art, similar to how any protein is coated to ELISA wells, for example. For Biacore, the dendrimer may be attached covalently through amine coupling to an amine chip using the protocol of the manufacturer, for example.

i. Multivalent Interactions

Multivalent interactions, in which multiple ligands of a single entity bind simultaneously to multiple receptors of another, occur throughout biology (e.g., virus-cell and bacterium-cell interactions) as reviewed in Mammen, 1998. Molecules that mimic or inhibit multivalent interactions can be used as potential therapeutics and/or diagnostic reagents. Identifying such molecules often results in lead compounds with relatively weak affinity for the target, however, much stronger interactions can be achieved from multivalent presentation of monovalent molecules (Carlson, 2007; Hong, 2007; Lee, 2000; Mammen, 1995; de Wildt, 2002; Gomara, 2000). Such multivalent presentations can vary in the number of molecules presented, for instance there could be 1 to 30, 1 to 20, 11-30, 11-20, 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 molecules presented in a multivalent interaction.

An illustrative type of multivalent interaction comprises peptide dendrimers. “Dendrimers” as used herein refers to repeatedly branched molecules. More specifically, dendrimers refer to branched polymers with peptides attached centrally to a core matrix (Lee, 2005). With the multiple antigen peptide (MAP) resin used for peptide synthesis, the peptide has to be the same in all branches. It may be possible to build custom resins with orthogonal protecting groups that would allow two different peptides to be made on one resin. The multiple antigenic peptide (MAP) system, which allows the assembly of multiple peptide sequences attached to a core of branching lysine residues with a defined structure, was introduced as a synthetic approach to the production of antigens (Niederhafner, 2005). Similar to multivalent interactions in general, described above, the number of branches of a dendrimer may vary. A dendrimer may be monovalent or multivalent for example, but not limited to, divalent, trivalent, tetravalent, pentavalent, hexavalent, heptavalent, octovalent, nanovalent, or decavalent. In other words, a dendrimer may have 1 to 30, 1 to 20, 11 to 30, 11 to 20, 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 branches. In a particular embodiment, a dendrimer is octovalent, having 8 branched polymers each comprising a peptide. In one embodiment, the dendrimer branches comprise the same peptide. In another embodiment, the branches of the dendrimer are different peptides. Dendrimers comprising branches of different proteins may be made by synthesizing branches of one protein, using an orthogonal protecting group to protect against polymerization, then synthesizing branches of another protein. This process may be repeated to create a dendrimer with branches comprising various different proteins. Examples of orthagonal protecting groups include, but are not limited to, Fmoc protective group, Boc protective group, benzyloxy-carbonyl (Z) group, allyloxycarbonly protecting group and lithographic protecting groups. It is contemplated that the dendrimer or the peptides of the dendrimer have the capacity to bind to a NoV or NoVLP. In one aspect of the invention, the dendrimer can bind to the NoV or NoVLP via one peptide of the dendrimer or via more than one peptide of the dendrimer. It is further contemplated that the peptides or dendrimers capable of binding a NoV or NoVLP are provided on a solid support structure such as a glass slide, a cellulose filter membrane, a bead, a column, a resin, a protein or other solid support structures that are commonly known in the art.

ii. Phage Display

Alternatively, initial screens can be performed with multivalent libraries, such as phage display libraries. Phage display has been shown to be an effective method for selecting peptides that bind to a target from large collections of random sequence peptides (Kehoe, 2005). A phage display library is a mixture of phage with foreign coding sequences spliced into the genome of the virion at one of the phage outer coat proteins (gene III, VI, VII, VIII, IX) (Kehoe, 2005). The peptide encoded by the foreign DNA is displayed on the surface of the virion as a fusion to one of the coat proteins. Each phage clone displays a single sequence of varying valency depending on which coat protein it is fused to (pIII, VI, VII, IX-5 copies; pVIII-2700 copies), with a phage library representing billions of multivalent peptide sequences (Kehoe, 2005; Cesareni, 1992; Noren, 2001; Petrenko, 2003; Sidhu, 2000; Hoess, 2001; Smith, 1997). Phage display has been successfully used to identify affinity reagents, enzyme inhibitors, artificial transcription factors, and protease substrates (Kehoe, 2005; Sidhu, 2000; Smith, 1997).

In particular, phage display can be used to identify peptides that bind to recombinant NoVLPs for development as a capture reagent of native NoVs. Thus, phage display random multi-mer peptide library can be used for biopanning to identify peptides that bind to NoVLPs, for instance genogroup I Norwalk VLPs (NVLPs). The multi-mer peptide library can include peptides that are 3 to 50 amino acids in length, or 3 to 20 amino acids in length, or 10 to 20 amino acids in length, or 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids in length.

After multiple rounds of panning, phage are then amplified from plaques from each round of panning, and binding of phage to immobilized VLPs can be evaluated by ELISA using a phage-specific antibody, for example. Clones that exhibited binding by ELISA can be further evaluated for binding to specific genogroup recombinant VLP strains and measured against nonspecific binding to negative control proteins. Finally, identified peptide sequences that exhibit binding to recombinant VLP strains can be synthesized and binding evaluated using surface plasmon resonance.

### V. Detecting Noroviruses

Included in one embodiment of the present invention are methods for detecting a NoV or NoVLP. The methods contemplated to achieve this involve the use of a peptide that has the capacity to bind to a NoV or NoVLP. Binding of the peptide to the NoV or NoVLP may then result in concentration of the bound virus or particle. The bound NoV or NoVLP can then be detected, several methods for detection are commonly known in the art, and can be employed in the present invention.

A. Immunodetection Methods

In one embodiment of the invention, immunodetection methods for binding, purifying, removing, quantifying, and/or otherwise generally detecting biological components such as a NoV or NoVLP, or a NoV or NoVLP bound to a peptide, or the complex of a NoV or NoVLP and the bound peptide. The steps of various useful immunodetection methods have been described in the scientific literature, such as, e.g., Doolittle M H and Ben-Zeev O, 1999; Gulbis B and Galand P, 1993; De Jager R et al., 1993; and Nakamura et al., 1987, each incorporated herein by reference. Some exemplary immunodetection methods include enzyme linked immunosorbent assay (ELISA), radioimmunoassay (RIA), immunoradiometric assay, fluoroimmunoassay, chemiluminescent assay, bioluminescent assay, and Western blot to mention a few.

Herein, the terms “antibody” and “immuologically active fragment” refer to any antibody-like molecule that has an antigen binding region, and includes antibody fragments.

In general, the immunobinding methods include obtaining a sample suspected of comprising virus, particle, protein, polypeptide and/or peptide, and contacting the sample with an antibody or an immunologically active fragment thereof to a NoV or NoVLP in accordance with the present invention, as the case may be, under conditions effective to allow the formation of immunocomplexes.

These methods include methods for purifying wild type and/or mutant viruses, particles, proteins, polypeptides and/or peptides as may be employed in purifying wild type and/or mutant viruses, particles, proteins, polypeptides and/or peptides from individuals' samples and/or for purifying recombinantly expressed wild type or mutant viruses, particles, proteins, polypeptides and/or peptides. In these instances, the antibody removes the antigenic wild type and/or mutant viruses, particles, proteins, polypeptides and/or peptides component from a sample. The antibody will preferably be linked to a solid support, such as in the form of a column matrix, and the sample suspected of containing the wild type or mutant protein antigenic component will be applied to the immobilized antibody. The unwanted components will be washed from the column, leaving the antigen immunocomplexed to the immobilized antibody, which wild type or mutant protein antigen is then collected by removing the wild type or mutant viruses, particles, proteins, polypeptides and/or peptides from the column.

The immunobinding methods also include methods for detecting and quantifying the amount of a wild type or mutant virus and/or protein reactive component in a sample and the detection and quantification of any immune complexes formed during the binding process. Here, one would obtain a sample suspected of comprising a wild type or mutant virus, protein and/or peptide and contact the sample with an antibody against wild type or mutant, and then detect and quantify the amount of immune complexes formed under the specific conditions.

In terms of antigen detection, the biological sample analyzed may be any sample that is suspected of containing a wild type or mutant protein-specific antigen, such as a specimen, a homogenized tissue extract, a cell, separated and/or purified forms of any of the above wild type or mutant virus or peptide-containing compositions.

Contacting the chosen biological sample with the antibody or immunologically active fragment under effective conditions and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to, any antigens present. After this time, the sample-antibody composition, such as a tissue section, ELISA plate, dot blot or western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, colorometric, biological and enzymatic tags. U.S. Patents concerning the use of such labels include U.S. Pat. Nos. 3,817,837; 3,850,752; 3,939,350; 3,996,345; 4,277,437; 4,275,149 and 4,366,241, each incorporated herein by reference. Of course, one may find additional advantages through the use of a secondary binding ligand such as a second antibody and/or a biotin/avidin ligand binding arrangement, as is known in the art.

The antibody employed in the detection may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two step approach. A second binding ligand, such as an antibody, that has binding affinity for the antibody is used to form secondary immune complexes, as described above. After washing, the secondary immune complexes are contacted with a third binding ligand or antibody that has binding affinity for the second antibody, again under effective conditions and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

The immunodetection methods of the present invention have evident utility in the diagnosis of conditions such as various forms of NoV infection. Here, a biological and/or clinical sample suspected of containing a wild type or mutant virus, particle, protein, polypeptide, peptide and/or mutant is used. However, these embodiments also have applications to non-clinical samples, such as in the titering of antigen or antibody samples, for example in the selection of hybridomas.

i. ELISAs

As detailed above, immunoassays, in their most simple and/or direct sense, are binding assays. Particular immunoassays are the various types of enzyme linked immunosorbent assays (ELISAs) and/or radioimmunoassays (RIA) known in the art. Immunohistochemical detection using tissue sections is also particularly useful. However, it will be readily appreciated that detection is not limited to such techniques, western blotting, dot blotting, FACS analyses, or the like may also be used.

In one exemplary ELISA, antibodies are immobilized onto a selected surface exhibiting protein affinity, such as a well in a polystyrene microtiter plate. Then, a test composition suspected of containing the wild type and/or mutant protein antigen, such as a clinical sample, is added to the wells. After binding and/or washing to remove non-specifically bound immune complexes, the bound wild type and/or mutant protein antigen may be detected. Detection is generally achieved by the addition of another antibody that is linked to a detectable label. This type of ELISA is a simple “sandwich ELISA.” Detection may also be achieved by the addition of a second antibody, followed by the addition of a third antibody that has binding affinity for the second antibody, with the third antibody being linked to a detectable label.

In another exemplary ELISA, the samples suspected of containing the wild type and/or mutant protein antigen are immobilized onto the well surface and/or then contacted with the antibodies of the invention. After binding and/or washing to remove non-specifically bound immune complexes, the bound antibodies are detected. Where the initial antibodies are linked to a detectable label, the immune complexes may be detected directly. Again, the immune complexes may be detected using a second antibody that has binding affinity for the first antibody, with the second antibody being linked to a detectable label.

Another ELISA in which the wild type and/or mutant proteins, polypeptides and/or peptides may be immobilized, involves the use of antibody competition in the detection. In this ELISA, labeled antibodies against wild type or mutant protein are added to the wells, allowed to bind, and/or detected by means of their label. The amount of wild type or mutant protein antigen in an unknown sample is then determined by mixing the sample with the labeled antibodies against wild type and/or mutant before and/or during incubation with coated wells. The presence of wild type and/or mutant protein in the sample acts to reduce the amount of antibody against wild type or mutant protein available for binding to the well and thus reduces the ultimate signal. This is also appropriate for detecting antibodies against wild type or mutant protein in an unknown sample, where the unlabeled antibodies bind to the antigen-coated wells and also reduces the amount of antigen available to bind the labeled antibodies.

Irrespective of the format employed, ELISAs have certain features in common, such as coating, incubating and binding, washing to remove non-specifically bound species, and detecting the bound immune complexes. These are described below.

In coating a plate with either antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein or solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface is contacted with the biological sample to be tested under conditions effective to allow immune complex (antigen/antibody) formation. Detection of the immune complex then requires a labeled secondary binding ligand or antibody, and a secondary binding ligand or antibody in conjunction with a labeled tertiary antibody or a third binding ligand.

“Under conditions effective to allow immune complex (antigen/antibody) formation” means that the conditions may include diluting the antigens and/or antibodies with solutions such as BSA, bovine gamma globulin (BGG) or phosphate buffered saline (PBS)/Tween. These added agents also tend to assist in the reduction of nonspecific background. Immune complex formation may also occur in the absense of such additional solutions.

The “suitable” conditions also mean that the incubation is at a temperature or for a period of time sufficient to allow effective binding. Incubation steps are typically from about 1 to 2 to 4 hours or so, at temperatures preferably on the order of 25° C. to 27° C., or may be overnight at about 4° C. or so.

Following all incubation steps in an ELISA, the contacted surface is washed so as to remove non-complexed material. A particular washing procedure includes washing with a solution such as PBS/Tween, or borate buffer. Following the formation of specific immune complexes between the test sample and the originally bound material, and subsequent washing, the occurrence of even minute amounts of immune complexes may be determined.

To provide a detecting means, the second or third antibody will have an associated label to allow detection. Preferably, this will be an enzyme that will generate color development upon incubating with an appropriate chromogenic substrate. Thus, for example, one may desire to contact or incubate the first and second immune complex with a urease, glucose oxidase, alkaline phosphatase or hydrogen peroxidase-conjugated antibody for a period of time and under conditions that favor the development of further immune complex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea, or bromocresol purple, or 2,2′-azino-di-(3-ethyl-benzthiazoline-6-sulfonic acid (ABTS), or H₂O₂, in the case of peroxidase as the enzyme label. Quantification is then achieved by measuring the degree of color generated, e.g., using a visible spectra spectrophotometer.

ii. Immunohistochemistry

The antibodies of the present invention may also be used in conjunction with both fresh-frozen and/or formalin-fixed, paraffin-embedded tissue blocks prepared for study by immunohistochemistry (IHC). The method of preparing tissue blocks from specimens has been successfully used in previous IHC studies of various prognostic factors, and is well known to those of skill in the art (Brown et al., 1990; Abbondanzo et al., 1990; Allred et al., 1990).

Briefly, frozen-sections may be prepared by rehydrating 50 ng of frozen “pulverized” tissue at room temperature in phosphate buffered saline (PBS) in small plastic capsules; pelleting the particles by centrifugation; resuspending them in a viscous embedding medium (OCT); inverting the capsule and pelleting again by centrifugation; snap-freezing in 70° C. isopentane; cutting the plastic capsule and removing the frozen cylinder of tissue; securing the tissue cylinder on a cryostat microtome chuck; and/or cutting 25-50 serial sections.

Permanent-sections may be prepared by a similar method involving rehydration of the 50 mg sample in a plastic microfuge tube; pelleting; resuspending in 10% formalin for 4 hours fixation; washing/pelleting; resuspending in warm 2.5% agar; pelleting; cooling in ice water to harden the agar; removing the tissue/agar block from the tube; infiltrating and/or embedding the block in paraffin; and cutting up to 50 serial permanent sections.

B. Immunodetection Kits

In still further embodiments, the present invention concerns immunodetection kits for use with the immunodetection methods described above. The immunodetection kits will comprise, in suitable container means, a peptide or polypeptide capable of binding a NoV or NoVLP, a first antibody that binds to a wild type or mutant virus, particle, peptide or polypeptide or optionally, an immunodetection reagent and/or further optionally, a wild type or mutant protein, polypeptide or peptide. The provided NoV binding peptide may or may not be provided on a solid support structure and further, may or may not be provided as a dendrimer.

In particular embodiments, monoclonal antibodies will be used. In certain embodiments, the first antibody that binds to the wild type or mutant protein, polypeptide or peptide may be pre-bound to a solid support, such as a column matrix and/or well of a microtitre plate, for example.

The immunodetection reagents of the kit may take any one of a variety of forms, including those detectable labels that are associated with and/or linked to the given antibody. Detectable labels that are associated with and/or attached to a secondary binding ligand are also contemplated. Exemplary secondary ligands are those secondary antibodies that have binding affinity for the first antibody.

Further suitable immunodetection reagents for use in the present kits include the two-component reagent that comprises a secondary antibody that has binding affinity for the first antibody, along with a third antibody that has binding affinity for the second antibody, the third antibody being linked to a detectable label. As noted above, a number of exemplary labels are known in the art and all such labels may be employed in connection with the present invention.

The kits may further comprise a suitably aliquoted composition of the wild type and/or mutant virus, particle, protein, polypeptide and/or polypeptide, whether labeled and/or unlabeled, as may be used to prepare a standard curve for a detection assay. The kits may contain antibody-label conjugates either in fully conjugated form, in the form of intermediates, and/or as separate moieties to be conjugated by the user of the kit. The components of the kits may be packaged either in aqueous media or in lyophilized form.

The container means of the kits will be suitable housed and will generally include at least one vial, test tube, flask, bottle, syringe and/or other container means, into which the antibody may be placed, and/or suitably aliquoted. Where wild type or mutant virus, particle, protein, polypeptide and/or peptide, and/or a second and/or third binding ligand and/or additional component is provided, the kit will also generally contain a second, third and/or other additional container into which this ligand and/or component may be placed. The kits of the present invention will also typically include a means for containing the antibody, antigen, and/or any other reagent containers in close confinement for commercial sale. Such containers may include injection and/or blow-molded plastic containers into which the desired vials are retained.

### VI. Polypeptides and Polynucleotides

A. Polypeptides

The terms “gene product,” “polypeptide”, “peptide” or “protein” that binds a NoV or NoVLP refer to an amino acid sequence that include related compounds of the respective molecules that exhibit at least some biological activity in common with their native counterparts (i.e. binding to a NoV or NoVLP). Such related compounds include, but are not limited to, truncated polypeptides or polypeptides having fewer amino acids than the native polypeptide. The peptide sequences that bind NoVs or NoVLPs may include, but are not limited to, SEQ ID NO: 1, SEQ ID NO: 2, SEQ ID NO: 8, SEQ ID NO: 10, SEQ ID NO: 11, SEQ ID NO: 3, SEQ ID NO: 4 and a peptide containing Pro-Xaa-Xaa at the C-terminus. The term “C-terminus” or “C-terminal end” refers to the last one or more amino acids in the sequence, for example the last 1, 2, 3, 4, 5, 6, 7, 8, 9, 10, 11, 12, 13, 14, 15 or 16 amino acids, etc. make up the C-terminus.

As modifications and/or changes may be made in the structure of the polynucleotides, polypeptides, peptides and/or proteins according to the present invention, while obtaining molecules having similar or improved characteristics, such biologically functional equivalents are also encompassed within the present invention.

In particular embodiments of the present invention, the peptides or polypeptides are truncated versions of the native sequences and in some embodiments the truncation is an N-terminal truncation. The term “N-terminus” or “N-terminal end” refers to the first one or more amino acids in the sequence, for example the first 1, 2, 3, 4, 5, 6, 7, 8, 9 or 10 amino acids, etc. make up the N-terminus. These N-terminal truncated peptides maintain the conserved C-terminal amino acids of their native sequences (Pro-Xaa-Xaa). N-terminal variants include, but are not limited to, deletion and/or substitution of one or more N-terminal amino acid residues, for example 1, 2, 3, 4, 5, 6, 7, 8, 9, 10, 11, 12, 13, 14, 15 or 16 N-terminal amino acid residues, etc. Thus, N-terminal variants comprise at least deletions or truncations and/or substitutions of 1 to 16 N-terminal amino acid residues, for example.

i. Modified Polypeptides

The biological functional equivalent may comprise a polynucleotide that has been engineered to contain distinct sequences while at the same time retaining the capacity to encode a standard peptide having a binding capacity similar to that of the native or truncated peptides described above. In specific embodiments, the polypeptide being modified may be the amino acid sequence of SEQ ID NO: 1, SEQ ID NO: 2, SEQ ID NO: 8, SEQ ID NO: 10, SEQ ID NO: 11, SEQ ID NO: 3, SEQ ID NO: 4 or a peptide comprising Pro-Xaa-Xaa. In another specific embodiment the polypeptide being modified may be a fragment or truncation of SEQ ID NO: 1, SEQ ID NO: 2, SEQ ID NO: 8, SEQ ID NO: 10, SEQ ID NO: 11, SEQ ID NO: 3, SEQ ID NO: 4 or a peptide comprising Pro-Xaa-Xaa.

This can be accomplished to the degeneracy of the genetic code, i.e., the presence of multiple codons, which encode for the same amino acids. In one example, one of skill in the art may wish to introduce a restriction enzyme recognition sequence into a polynucleotide while not disturbing the ability of that polynucleotide to encode a peptide or protein.

In another example, a polynucleotide may be (and encode) a biological functional equivalent with more significant changes. Certain amino acids may be substituted for other amino acids in a peptide or protein structure without appreciable loss of interactive binding capacity and with the potential to increase binding capacity. So-called “conservative” changes do not disrupt the biological activity of the peptide or protein, as the structural change is not one that impinges of the peptide's or protein's ability to carry out its designed function. It is thus contemplated by the inventors that various changes may be made in the sequence of genes and peptides disclosed herein, while still fulfilling the goals of the present invention.

In terms of functional equivalents, it is well understood by the skilled artisan that, inherent in the definition of a “biologically functional equivalent” peptide and/or polynucleotide, is the concept that there is a limit to the number of changes that may be made within a defined portion of the molecule while retaining a molecule with an acceptable level of equivalent biological activity. Biologically functional equivalents are thus defined herein as those peptides or proteins (and polynucleotides) in which selected amino acids (or codons) may be substituted. Functional activity is defined as the ability to bind to NoVs or NoVLPs. Thus, the function equivalent is a peptide that is capable of binding to a NoV or NoVLP.

Amino acid substitutions are generally based on the relative similarity of the amino acid side-chain substituents, for example, their hydrophobicity, hydrophilicity, charge, size, and/or the like. An analysis of the size, shape and/or type of the amino acid side-chain substituents reveals that arginine, lysine and histidine are all positively charged residues; that alanine, glycine and serine are all a similar size; and that phenylalanine, tryptophan and tyrosine all have a generally similar shape. Therefore, based upon these considerations, arginine, lysine and histidine; alanine, glycine and serine; and phenylalanine, tryptophan and tyrosine; are defined herein as biologically functional equivalents.

To effect more quantitative changes, the hydropathic index of amino acids may be considered. Each amino acid has been assigned a hydropathic index on the basis of their hydrophobicity and/or charge characteristics, these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and/or arginine (−4.5).

The importance of the hydropathic amino acid index in conferring interactive biological function on a peptide or protein is generally understood in the art (Kyte and Doolittle, 1982, incorporated herein by reference). It is known that certain amino acids may be substituted for other amino acids having a similar hydropathic index and/or score and/or still retain a similar biological activity. In making changes based upon the hydropathic index, the substitution of amino acids whose hydropathic indices are within ±2 is preferred, those which are within ±1 are particularly preferred, and/or those within ±0.5 are even more particularly preferred.

It also is understood in the art that the substitution of like amino acids can be made effectively on the basis of hydrophilicity. As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). In making changes based upon similar hydrophilicity values, the substitution of amino acids whose hydrophilicity values are within ±2 is preferred, those which are within ±1 are particularly preferred, and those within ±0.5 are even more particularly preferred.

ii. Altered Amino Acids

The present invention, may rely on the synthesis of peptides and polypeptides in cyto, via transcription and translation of appropriate polynucleotides. These peptides and polypeptides will include the twenty “natural” amino acids, and post-translational modifications thereof. However, in vitro peptide synthesis, also relied upon in the present invention, permits the use of modified and/or unusual amino acids. A table of exemplary, but not limiting, modified and/or unusual amino acids is provided herein below in Table 1.

TABLE 1
Modified and/or Unusual Amino Acids

| Abbr. | Amino Acid | Abbr. | Amino Acid |
|---|---|---|---|
| Aad | 2-Aminoadipic acid | EtAsn | N-Ethylasparagine |
| BAad | 3-Aminoadipic acid | Hyl | Hydroxylysine |
| BAla | beta-alanine, beta-Amino-propionic acid | AHyl | allo-Hydroxylysine |
| Abu | 2-Aminobutyric acid | 3Hyp | 3-Hydroxyproline |
| 4Abu | 4-Aminobutyric acid, piperidinic acid | 4Hyp | 4-Hydroxyproline |
| Acp | 6-Aminocaproic acid | Ide | Isodesmosine |
| Ahe | 2-Aminoheptanoic acid | Aile | allo-Isoleucine |
| Aib | 2-Aminoisobutyric acid | MeGly | N-Methylglycine, sarcosine |
| BAib | 3-Aminoisobutyric acid | MeIle | N-Methylisoleucine |
| Apm | 2-Aminopimelic acid | MeLys | 6-N-Methyllysine |
| Dbu | 2,4-Diaminobutyric acid | MeVal | N-Methylvaline |
| Des | Desmosine | Nva | Norvaline |
| Dpm | 2,2′-Diaminopimelic acid | Nle | Norleucine |
| Dpr | 2,3-Diaminopropionic acid | Orn | Ornithine |
| EtGly | N-Ethylglycine | | |

iii. Synthetic Peptides

In some embodiments the NoV binding peptides are short peptides, such as, but not limited to, peptides equal to or shorter than 50 amino acids in length. For instance, peptides equal to or less than 50, 45, 40, 35, 30, 29, 28, 27, 26, 25, 24, 23, 22, 21, 20, 19, 18, 17, 16, 15, 14, 13, 12, 11, 10, 9, 8, 7, 6, 5, or 4 amino acids in length. Because of their relatively small size, the peptides of the invention can also be synthesized in solution or on a solid support in accordance with conventional techniques. Various automatic synthesizers are commercially available and can be used in accordance with known protocols. See, for example, Stewart and Young (1984); Tam et al. (1983); Merrifield (1986); and Barany and Merrifield (1979), each incorporated herein by reference. Short peptide sequences, or libraries of overlapping peptides, usually from about 3 up to about 35 to 50 amino acids, can be readily synthesized and then screened in screening assays designed to identify reactive peptides. Alternatively, recombinant DNA technology may be employed wherein a nucleotide sequence that encodes a peptide of the invention is inserted into an expression vector, transformed or transfected into an appropriate host cell, and cultivated under conditions suitable for expression.

B. Polynucleotides

The term “polynucleotide,” “oligonucleotide,” or “nucleic acid” refers to at least one molecule or strand of DNA (e.g., genomic DNA, cDNA) or RNA sequence (antisense RNA, siRNA, shRNA) a derivative or mimic thereof, comprising at least one nucleotide base, such as, for example, a naturally occurring purine or pyrimidine base found in DNA (e.g., adenine “A,” guanine “G,” thymine “T,” and cytosine “C”) or RNA (e.g., A, G, uracil “U,” and C). The term “nucleic acid” encompasses the terms “oligonucleotide” and “polynucleotide.” These definitions generally refer to at least one single-stranded molecule, but in specific embodiments will also encompass at least one additional strand that is partially, substantially or fully complementary to the at least one single-stranded molecule. Thus, a nucleic acid may encompass at least one double-stranded molecule or at least one triple-stranded molecule that comprises one or more complementary strand(s) or “complement(s)” of a particular sequence comprising a strand of the molecule. An “isolated nucleic acid” as contemplated in the present invention may comprise transcribed nucleic acid(s), regulatory sequences, coding sequences, or the like, isolated substantially away from other such sequences, such as other naturally occurring nucleic acid molecules, regulatory sequences, polypeptide or peptide encoding sequences, etc.

More particularly, a “gene” or “polynucleotide” may also comprise a DNA or RNA sequence which encodes a peptide with the capacity to bind a NoV or NoVLP. These terms may further comprise any combination of associated control sequences, as well related sequences, such as fragments, of the respective molecules that exhibit at least some biological activity in common with their native counterparts. Biological activity similar to the native counterparts will include coding for a peptide that has activity that binds to NoV or NoVLPs. The polynucleotide sequences contemplated include, but are not limited to polynucleotide sequences that encode an amino acid sequence of SEQ ID NO: 1, SEQ ID NO: 2, SEQ ID NO: 8, SEQ ID NO: 10, SEQ ID NO: 11, SEQ ID NO: 3, SEQ ID NO: 4 and a peptide containing Pro-Xaa-Xaa, and based on table 2 one skilled in the art would easily be able to determine these sequences.

Yet further, nucleic acid sequences of the present invention can relate to the truncated peptides having increased NoV or NoVLP binding activity compared to the native sequences.

It also is contemplated that a given peptide or protein may be represented by variants, that have slightly different nucleic acid sequences but, nonetheless, encode the same peptide or protein (see Table 2 below). The nucleic acid sequences may be naturally occurring or synthetic.

TABLE 2

| Amino Acids | | | Codons |
|---|---|---|---|
| Alanine | Ala | A | GCA GCC GCG GCU |
| Cysteine | Cys | C | UGC UGU |
| Aspartic acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

Still further, the “nucleic acid sequence,” “polynucleotide,” and “gene product” refer to nucleic acids provided herein, analogs thereof, homologs thereof, and sequences having substantial similarity and function, respectively. The term “substantially identical”, when used to define either a peptide that binds to a NoV or NoVLP amino acid sequence or the polynucleotide sequence encoding said peptide, means that a particular subject sequence, for example, a mutant sequence, varies from the sequence of said NoV binding peptide or the polynucleotide encoding it, respectively, by one or more substitutions, deletions, or additions, the net effect of which is to retain at least some of the biological activity found in the native peptide, respectively. Alternatively, DNA analog sequences are “substantially identical” to specific DNA sequences disclosed herein if: (a) the DNA analog sequence is derived from coding regions of the polynucleotide encoding the NoV binding peptide, respectively; or (b) the DNA analog sequence is capable of hybridization to DNA sequences of (a) under moderately stringent conditions and, respectively having biological activity similar to the native peptides; or (c) DNA sequences which are degenerative as a result of the genetic code to the DNA analog sequences defined in (a) or (b). Substantially identical analog peptides will be greater than about 80% similar to the corresponding sequence of the full-length native peptide, more preferably, greater than 90% similar to the corresponding sequence of the full-length native peptide, and most preferably, greater than 95% to the corresponding sequence of the full-length native peptide. Sequences having lesser degrees of similarity but comparable biological activity are considered to be equivalents. Comparable biological activity would include the ability to produce peptide or polypeptide that is capable of binding a NoV or NoVLP. In determining polynucleotide sequences, all subject polynucleotide sequences capable of encoding substantially similar amino acid sequences are considered to be substantially similar to a reference polynucleotide sequence, regardless of differences in codon sequence.

As used herein, “hybridization,” “hybridizes,” or “capable of hybridizing” is understood to mean the forming of a double or triple stranded molecule or a molecule with partial double or triple stranded nature. The term “hybridization,” “hybridize(s),” or “capable of hybridizing” encompasses the terms “stringent condition(s)” or “high stringency” and the terms “low stringency” or “low stringency condition(s)” or “moderately stringent conditions.”

As used herein “stringent condition(s)” or “high stringency” are those conditions that allow hybridization between or within one or more nucleic acid strand(s) containing complementary sequence(s), but precludes hybridization of random sequences. Stringent conditions tolerate little, if any, mismatch between a nucleic acid and a target strand. Such conditions are well known to those of ordinary skill in the art, and are preferred for applications requiring high selectivity. Non-limiting applications include isolating a nucleic acid, such as a gene or a nucleic acid segment thereof, or detecting at least one specific mRNA transcript or a nucleic acid segment thereof, and the like.

Stringent conditions may comprise low salt and/or high temperature conditions, such as provided by about 0.02 M to about 0.15 M NaCl at temperatures of about 50° C. to about 70° C. It is understood that the temperature and ionic strength of a desired stringency are determined in part by the length of the particular nucleic acid(s), the length and nucleobase content of the target sequence(s), the charge composition of the nucleic acid(s), and to the presence or concentration of formamide, tetramethylammonium chloride or other solvent(s) in a hybridization mixture.

It is also understood that these ranges, compositions and conditions for hybridization are mentioned by way of non-limiting examples only, and that the desired stringency for a particular hybridization reaction is often determined empirically by comparison to one or more positive or negative controls. Depending on the application envisioned it is preferred to employ varying conditions of hybridization to achieve varying degrees of selectivity of a nucleic acid towards a target sequence. In a non-limiting example, identification or isolation of a related target nucleic acid that does not hybridize to a nucleic acid under stringent conditions may be achieved by hybridization at low temperature and/or high ionic strength. For example, a medium or moderate stringency condition could be provided by about 0.1 to 0.25 M NaCl at temperatures of about 37° C. to about 55° C. Under these conditions, hybridization may occur even though the sequences of probe and target strand are not perfectly complementary, but are mismatched at one or more positions. In another example, a low stringency condition could be provided by about 0.15 M to about 0.9 M salt, at temperatures ranging from about 20° C. to about 55° C. Of course, it is within the skill of one in the art to further modify the low or high stringency conditions to suit a particular application. For example, in other embodiments, hybridization may be achieved under conditions of, 50 mM Tris-HCl (pH 8.3), 75 mM KCl, 3 mM MgCl₂, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C.

Naturally, the present invention also encompasses nucleic acid sequences that are complementary, or essentially complementary, to the sequences set forth herein. Nucleic acid sequences that are “complementary” are those that are capable of base-pairing according to the standard Watson-Crick complementarity rules. As used herein, the terms “complementary sequences” and “essentially complementary sequences” means nucleic acid sequences that are substantially complementary to, as may be assessed by the same nucleotide comparison set forth above, or are able to hybridize to a nucleic acid segment of one or more sequences set forth herein. Such sequences may encode an entire peptide or functional or non-functional fragments thereof.

In certain embodiments, a “complementary” nucleic acid comprises a sequence in which about 70%, about 71%, about 72%, about 73%, about 74%, about 75%, about 76%, about 77%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, to about 100%, and any range derivable therein, of the nucleobase sequence is capable of base-pairing with a single or double stranded nucleic acid molecule during hybridization. In certain embodiments, the term “complementary” refers to a nucleic acid that may hybridize to another nucleic acid strand or duplex in stringent conditions, as would be understood by one of ordinary skill in the art.

In certain embodiments, a “partly complementary” nucleic acid comprises a sequence that may hybridize in low stringency conditions to a single or double stranded nucleic acid, or contains a sequence in which less than about 70% of the nucleobase sequence is capable of base-pairing with a single or double stranded nucleic acid molecule during hybridization.

C. Expression Vectors

The present invention may involve using expression constructs as the pharmaceutical compositions. In certain embodiments, it is contemplated that the expression construct comprises one or more polynucleotide sequences encoding polypeptides which have the capacity to bind to NoVs or NoVLPs.

As used herein, the term “expression vector” refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules or ribozymes or siRNA. Expression vectors can contain a variety of control sequences, which refer to nucleic acid sequences necessary for the transcription and possibly translation of an operatively linked coding sequence in a particular host organism. In addition to control sequences that govern transcription and translation, vectors and expression vectors may contain nucleic acid sequences that serve other functions as well and are described infra. One skilled in the art realizes that the present invention utilizes the expression vector as a therapy to treat acute gastroenteritis caused by for example NoV infection. The expression vector may also be used to produce large amount of the peptide for attachment to solid surfaces, for example.

In particular embodiments of the invention, a plasmid vector is contemplated for use to transform a host cell. In general, plasmid vectors containing replicon and control sequences that are derived from species compatible with the host cell are used in connection with these hosts. The vector ordinarily carries a replication site, as well as marking sequences that are capable of providing phenotypic selection in transformed cells. Plasmid vectors are well known and are commercially available. Such vectors include, but are not limited to, pWLNEO, pSV2CAT, pOG44, PXT1, pSG (Stratagene) pSVK3, pBSK, pBR322, pUC vectors, vectors that contain markers that can be selected in mammalian cells, such as pcDNA3.1, episomally replicating vectors, such as the pREP series of vectors, pBPV, pMSG, pSVL (Pharmacia), adenovirus vector (AAV; pCWRSV, Chatterjee et al. (1992); retroviral vectors, such as the pBABE vector series, a retroviral vector derived from MoMuLV (pGlNa, Zhou et al., (1994); and pTZ18U (BioRad, Hercules, Calif.).

In one embodiment, a gene encoding a NoV or NoVLP binding peptide is introduced in vivo in a viral vector. The ability of certain viruses to enter cells via receptor-mediated endocytosis and to integrate into the host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986). Such vectors include an attenuated or defective DNA virus, such as but not limited to herpes simplex virus (HSV), papilloma virus, Epstein Barr virus (EBV), adenovirus, adeno-associated virus (AAV), lentivirus, polyoma virus, hepatitis B virus and the like. Defective viruses, which entirely or almost entirely lack viral genes, are preferred. Defective virus is not infective after introduction into a cell. Use of defective viral vectors allows for administration to cells in a specific, localized area, without concern that the vector can infect other cells. Thus, any tissue can be specifically targeted. Examples of particular vectors include, but are not limited to, a defective herpes virus 1 (HSV1) vector (Kaplitt et al., 1991) an attenuated adenovirus vector, (Stratford-Perricaudet et al., 1992), and a defective adeno-associated virus vector (Samulski et al., 1987 and Samulski et al., 1989).

In another embodiment the gene can be introduced in a retroviral vector, e.g., as described in U.S. Pat. No. 5,399,346; Mann et al., 1983; U.S. Pat. No. 4,650,764; U.S. Pat. No. 4,980,289; Markowitz et al., 1988; U.S. Pat. No. 5,124,263; International Patent Publication No. WO 95/07358; and Kuo et al., 1993, each of which is incorporated herein by reference in its entirety. Targeted gene delivery is described in International Patent Publication WO 95/28494.

Alternatively, the vector can be introduced in vivo by lipofection. For the past decade, there has been increasing use of liposomes for encapsulation and transfection of nucleic acids in vitro. Synthetic cationic lipids designed to limit the difficulties and dangers encountered with liposome mediated transfection can be used to prepare liposomes for in vivo transfection of a gene encoding a marker. The use of cationic lipids may promote encapsulation of negatively charged nucleic acids, and also promote fusion with negatively charged cell membranes. The use of lipofection to introduce exogenous genes into the specific organs in vivo has certain practical advantages. Molecular targeting of liposomes to specific cells represents one area of benefit. Lipids may be chemically coupled to other molecules for the purpose of targeting. Targeted peptides, e.g., hormones or neurotransmitters, and proteins such as antibodies, or non-peptide molecules could be coupled to liposomes chemically.

It is also possible to introduce the vector in vivo as a naked DNA plasmid. Naked DNA vectors for gene therapy can be introduced into the desired host cells by methods known in the art, e.g., transfection, electroporation, microinjection, transduction, cell fusion, DEAE dextran, calcium phosphate precipitation, use of a gene gun, or use of a DNA vector transporter (Wu and Wu, 1988).

As used herein, the terms “cell,” “cell line,” and “cell culture” may be used interchangeably. One of skill in the art is cognizant that in addition to commercially available cell lines, primary cultures of cells may also be used in the present invention. All of these terms also include their progeny, which is any and all subsequent generations formed by cell division. It is understood that all progeny may not be identical due to deliberate or inadvertent mutations. A host cell may be “transfected” or “transformed,” which refers to a process by which exogenous nucleic acid is transferred or introduced into the host cell. A transformed cell includes the primary subject cell and its progeny. As used herein, the terms “engineered” and “recombinant” cells or host cells are intended to refer to a cell into which an exogenous nucleic acid sequence, such as, for example, a nucleic acid sequence encoding a peptide with the capacity to bind to a NoV or NoVLP. Therefore, recombinant cells are distinguishable from naturally occurring cells that do not contain a recombinantly introduced nucleic acid.

In certain embodiments, it is contemplated that nucleic acid or proteinaceous sequences may be co-expressed with other selected nucleic acid or proteinaceous sequences in the same host cell. Co-expression may be achieved by co-transfecting the host cell with two or more distinct recombinant vectors. Alternatively, a single recombinant vector may be constructed to include multiple distinct coding regions for nucleic acids, which could then be expressed in host cells transfected with the single vector.

### VI. Treatment/Prevention of a Norovirus Infection

In certain aspects of the present invention, compounds are used to treat and/or prevent acute gastroenteritis due to a NoV infection. More particularly, the compounds comprise a peptide that has the capacity to bind to a NoV or NoVLP. The peptide may or may not be provided on a solid support structure and further may or may not be provided as a dendrimer.

An individual to be treated using the NoV binding peptide can be an individual suffering from acute gastroenteritis or a NoV infection, or an individual who is known or suspected of being free of a NoV infection at the time peptide is administered. The individual, for example, can be an individual with no known disease or health-related condition (i.e., a healthy individual). In some embodiments, the individual is an individual at risk of developing. acute gastroenteritis or a NoV infection. Thus, in certain embodiments of the invention, methods include identifying an individual in need of treatment. An individual may be identified, for example, based on taking an individual history, or based on findings on clinical examination. An individual may be any vertebrate, such as a mammal. In particular embodiments, the individual is a human.

Treatment and/or prevention methods will involve treating an individual with an effective amount of a composition containing a NoV binding peptide. An effective amount is described, generally, as that amount sufficient to detectably and repeatedly to ameliorate, reduce, minimize or limit the extent of a disease or its symptoms. In the context of prevention, an effective amount is generally an amount that is sufficient to block the onset of a disease or its symptoms.

Prevention of NoV or NoVLP infection involves taking preventative measures, delivering a peptide that binds to the NoV or NoVLP for instance, especially when individuals who are susceptible to a NoV or NoVLP infection are concerned. In certain aspects, individuals susceptible to a NoV or NoVLP infection comprise people who are exposed to a large number of individuals, for instance, but not limited to, while traveling (on a plane, or a cruise ship or any other form of traveling), in a medical facility (either as a patient, worker or visitor), in a school (either as a teacher, staff, student or parent) or any other gathering of individuals. In some embodiments, an individuals who meet the criteria above may be an individual in need of preventative therapy, although, preventative therapy is not limited to use in only those individuals meeting the above criteria.

### VII. Treatment and/or Prevention Kits

In still further embodiments, the present invention concerns treatment and/or prevention kits for use relating to the treatment and prevention methods described above. The treatment and/or prevention kits will comprise, in suitable container means, a peptide or polypeptide capable of binding a NoV or NoVLP. The provided NoV binding peptide may or may not be provided on a solid support structure and further, may or may not be provided as a dendrimer.

Other objects, features and advantages of the present invention will become apparent from the following detailed description. It should be understood, however, that the detailed description and the specific examples, while indicating particular embodiments of the invention, are given by way of illustration only, since various changes and modifications within the spirit and scope of the invention will become apparent to those skilled in the art from this detailed description.

### EXAMPLES

The following examples are included to demonstrate particular embodiments of the invention. It should be appreciated by those of skill in the art that the techniques disclosed in the examples which follow represent techniques discovered by the inventor to function well in the practice of the invention, and thus can be considered to constitute particular modes for its practice. However, those of skill in the art should, in light of the present disclosure, appreciate that many changes can be made in the specific embodiments which are disclosed and still obtain a like or similar result without departing from the spirit and scope of the invention.

### Example 1

### Phage Display

The Ph.D.-12 peptide library (New England Biolabs) was used to identify peptides that bound to immobilized Norwalk VLPs (NVLPs). The Ph.D.-12 library consists of random sequence 12-mers fused to the minor coat protein (pIII) of M13 phage. Three rounds of biopanning was performed according to the manufacturer's instructions.

### Example 2

### Phage ELISA

Ten phage from each of the three rounds of panning were amplified according to the Ph.D.-12 Phage Display Peptide Library Kit protocol (New England Biolabs). Wells of a microplate were coated with 100 μL of NVLPs (10 μg/mL) in phosphate buffered saline (PBS) overnight at 4° C. and blocked with 5% nonfat milk in PBS containing 0.1% Tween-20 (v/v) (PBST) for 1 hour at room temperature. The amplified phage (25 μL) was diluted to 200 μL in PBST and added to the coated wells and incubated 2 hours at room temperature. The wells were washed with PBST (6×200 μL). Phage that bound NVLPs were detected with the anti-M13-HRP antibody (Amersham Biosciences) and the HRP substrate 2,2′-azinobis[3-ethylbenzthiazoline-6-sulfonic acid] diammonium salt (ABTS) with detection at 405 nm. M13 phage without a peptide fused to the coat protein was used as a control. The phage that showed a signal higher than 0.1 were tested by ELISA for binding to the genogroup II Houston, Grimsby, and Snow Mountain Norovirus-like particles coated at 10 μg/mL (100 μL) and for nonspecific binding to the following proteins: alcohol dehydrogenase, β-amylase, carbonic anhydrase, and green fluorescent protein, each coated at 10 μg/mL (100 μL).

### Example 3

### Peptide Synthesis

Peptides were synthesized by solid phase peptide synthesis using an Intavis Bioanalytical Instruments AG MultiPep peptide synthesizer using Fmoc chemistry on Tentagel amide resin (0.26 mmol/g, Intavis) or Fmoc-8-branch MAPS resin (0.55 mmol/g, Anaspec). The synthesis scale was 0.025 mmol and couplings were done with 1 equivalent of Fmoc-amino acid, 1 equivalent N-hydroxybenzotriazole (HOBt), and 2 equivalents of N,N′-dicyclohexylcarbodiimide (DIC). A 20% piperidine solution in DMF was used for the deprotection cycles. Biotinylation of amino groups was performed after peptide synthesis was complete. Biotin was activated (1 mmol in dimethylformamide (DMF)-dimethysulfoxide (DMSO) (1:1)) by addition of HOBt/HBTU (2-(1H-benzotriazole-1-yl)-1,1,3,3-tetramethyluronium hexafluorophosphate) and N,N-diisopropylethylamine. This solution was added to the resin and stirred overnight followed by washing with DMF-DMSO (2×), DMF (2×), and methylene chloride (2×). The resin was dried prior to cleavage. Peptides were fully deprotected and cleaved from the resins by treatment with 0.5 mL of a 95:2.5:2.5 (v/v) trifluoroacetic acid:triisopropyl silane:water cocktail or 80:10:10 (v/v) trifluoroacetic acid:1,2-ethanedithiol:thioanisole for cysteine-containing peptides for 2 hours at room temperature. The peptides were precipitated with cold diethyl ether and dried. Peptides were characterized by HPLC, MALDI-TOF MS, and electrospray MS.

### Example 4

### Peptide Conjugation

Peptides were conjugated to bovine serum albumin using the Imject Immunogen EDC Conjugation Kit (Pierce) according to the manufacturer's protocol. Peptides were coupled to the SulfoLink Coupling Gel (Pierce) according to the manufacturer's protocol.

### Example 5

### Dendrimer ELISA

Wells of a microplate were coated with 100 μL of RHFQTHTVPLSL₈ (the octovalent dendrimer of SEQ ID NO: 1), or AHFQTHTVPLSL₈ (the octovalent dendrimer of SEQ ID NO: 2) (0.4 mg/mL, 32 μM or 36 μM) in PBS overnight at 4° C. and blocked with 5% nonfat milk in PBST for 1 hour at room temperature. VLPs (Norwalk (NVLPs), Snow Mountain (SMV), Grimsby (GRV), and Houston (HOV)) (2 μg/mL, 100 μL) in PBST were added to the coated wells and incubated 1 hour at room temperature. The wells were washed with PBST (6×200 μL). Bound VLPs were detected with MAb3912 (Hardy, 1996). (NVLPs) or NS14 (Kitamoto, 2002). (SMV, GRV, HOV) as the primary antibody and goat anti-mouse-HRP as the secondary antibody with detection at 405 nm using the HRP substrate ABTS. Binding to wells coated with a nonspecific octovalent dendrimer, SREPQYGGPAAD₈ (the octovalent dendrimer of SEQ ID NO: 5) (0.4 mg/mL, 36 μM), was used as the negative control.

### Example 6

### Surface Plasmon Resonance

Peptide binding to VLPs was detected using a BIAcore 3000 instrument. VLPs were immobilized to a CM5 sensor chip using amine coupling at 5 μL/min in HBS buffer (10 mM HEPES containing 0.15 M NaCl and 0.005% surfactant P20, pH 7.4): the sensor chip surface was activated by a 10 min injection of a solution of 0.05 M NHS (N-hydroxysuccinimide) and 0.2 M EDC (N-ethyl-N′-dimethylaminopropylcarbodiimide) followed by a 5 min injection of VLPs (100 μg/mL) in 10 mM sodium acetate pH 4 and blocking of unreacted activated groups with a 7 min injection of ethanolamine. Final surface densities were typically around 9000 RU.

### Example 7

### Expression and Purification of NV P Domain-GST

A single colony of E. coli BL21 Gold harboring NVpGEX3-6 (Parker, 2005) was used to inoculate 50 mL of LB-ampicillin (100 μg/mL) and grown aerobically overnight at 37° C. with shaking. This entire culture was used to inoculate 1 L of LB-amp and growth with shaking continued at room temperature until A₆₀₀ ˜0.6. Protein expression was induced by addition of isopropyl-α-D-thiogalactopyranoside (1 mM), and after overnight incubation at room temperature with shaking, cells were harvested by centrifugation (5,000×g, 20 min, 4° C.). Cells were lysed using the BugBuster reagent (Novagen) in phosphate buffered saline according to the manufacturer's instructions and cell debris was removed by centrifugation (13,000×g, 20 min, 4° C.). The P-domain glutathione transferase fusion was purified using GST•bind resin (Novagen) according to the manufacturer's instructions. Approximately 7 mg of purified protein were obtained per liter of E. coli culture.

### Example 8

### Native NoV Capture and Detection in Clinical Samples

Capture and detection of native NoV from clinical samples was performed by ELISA. Microplate wells were coated with RHFQTHTVPLSL₈ (the octovalent dendrimer of SEQ ID NO: 1) (0.4 mg/mL, 32 μM, 100 μL), AHFQTHTVPLSL₈ (the octovalent dendrimer of SEQ ID NO: 2) (0.4 mg/mL, 36 μM, 100 μL), or an anti-Norwalk virus antibody from guinea pig (1:5000 dilution, 100 μL) in phosphate buffered saline (PBS) overnight at 4° C., blocked with 5% milk in PBS for 1 hour at room temperature, and washed with PBS-Tween-20 (0.05%, PBST, 6×200 μL). Stool samples (10% suspensions diluted 2-fold in 0.5% milk in PBS containing 1% goat serum (Sigma); 100 μL) were added to the coated wells and incubated for 1 hour at room temperature. The wells were washed with PBST (6×200 μL). Captured NoV was detected with an anti-Norwalk virus antibody from rabbit (for genogroup I Norwalk detection) or NS14 (for genogroup II NoV detection) as the primary antibody followed by goat anti-rabbit-HRP or goat anti-mouse-HRP as the secondary antibody with detection at 405 nm using the HRP substrate ABTS.

All of the compositions, methods and kits disclosed and claimed herein can be made and executed without undue experimentation in light of the present disclosure. While the compositions and methods of this invention have been described in terms of particular embodiments, it will be apparent to those of skill in the art that variations may be applied to the compositions, methods and kits and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

### REFERENCES

The following references, to the extent that they provide exemplary procedural or other details supplementary to those set forth herein, are specifically incorporated herein by reference.

### Patents and Patent Applications

U.S. Pat. No. 3,817,837
U.S. Pat. No. 3,850,752
U.S. Pat. No. 3,939,350
U.S. Pat. No. 3,996,345
U.S. Pat. No. 4,275,149
U.S. Pat. No. 4,277,437
U.S. Pat. No. 4,366,241
U.S. Pat. No. 4,650,764
U.S. Pat. No. 4,980,289
U.S. Pat. No. 5,124,263
U.S. Pat. No. 5,399,346
U.S. Pat. No. 7,205,112 B2
U.S. Patent Application No. 2005/0152911
U.S. Patent Application No. 2006/003957
International Publication No. WO 95/07358
International Publication No. WO 95/28494
Japanese Publication No. JP 2005/082558A

### PUBLICATIONS

Abbondanzo et al., Breast Cancer Res. Treat., 16: 182(#151), 1990.
Ando, T., Noel, J. S., and Fankhauser, R. L., “Genetic classification of Norwalk-like viruses,” J Infect Dis 181 Suppl 2, S336-348, 2000.
Allred et al., Breast Cancer Res. Treat., 16: 182(#149), 1990.
Baichwal and Sugden, In: GENE TRANSFER Kucherlapati, R., ed. New York: Plenum Press, pp. 117-148, 1986
Barany and Merrifield, “The Peptides, Gross and Meienhofer, eds”, Academic Press, New York, 1-284, 1979.
Batten, C A et al., Virology, 356(1-2):179-87. 2006.
Bertolotti-Ciarlet, A., White, L. J., Chen, R., Prasad, B. V., and Estes, M. K., “Structural requirements for the assembly of Norwalk virus-like particles,” J Virol 76, 4044-4055, 2002.
Bertolotti-Ciarlet, A., Crawford, S. E., Hutson, A. M., and Estes, M. K., The 3′ End of Norwalk Virus mRNA Contains Determinants That Regulate the Expression and Stability of the Viral Capsid ProteinVP1: a Novel Function for the VP2 Protein, J. Virol 77(21), 11603-11615.
Blanton, L. H., Adams, S. M., Beard, R. S., Wei, G., Bulens, S. N., Widdowson, M. A., Glass, R. I., and Monroe, S. S., “Molecular and Epidemiologic Trends of Caliciviruses Associated with Outbreaks of Acute Gastroenteritis in the United States 2000-2004,” J Infect Dis 193, 413-421, 2006.
Brown et al., Breast Cancer Res. Treat., 16: 192(#191), 1990.
Bull, R. A., Tu, E. T., McIver, C. J., Rawlinson, W. D., and White, P. A., “Emergence of a new norovirus genotype II.4 variant associated with global outbreaks of gastroenteritis,” J Clin Microbiol 44, 327-333, 2006.
Carillo, H., et al., SIAM, J Applied Math., 48:1073, 1988.
Carlson, C. B., Mowery, P., Owen, R. M., Dykhuizen, E. C., and Kiessling, L. L., “Selective tumor cell targeting using low-affinity, multivalent interactions,” ACS Chem Biol 2, 119-127, 2007.
Cesareni, G., “Peptide display on filamentous phage capsids: A new powerful tool to study protein-ligand interaction,” FEBS Lett 307, 66-70, 1992.
Chatterjee S, Johnson P R, Wong K K Jr., “Dual-target inhibition of HIV-1 in vitro by means of an adeno-associated virus antisense vector,” Science, November 27; 258(5087):1485-8, 1992.
De Jager R, et al., Semin Nucl Med 23(2):165-179, 1993.
Devereux, J., et al., Nucleic Acids Research, 12(1), 387, 1984.
de Wildt, R. M., Tomlinson, I. M., Ong, J. L., and Holliger, P., “Isolation of receptor-ligand pairs by capture of long-lived multivalent interaction complexes,” Proc Natl Acad Sci USA 99, 8530-8535, 2002.
Doolittle M. H. et al., Methods Mol. Biol., 109:215-237, 1999.
Gomara, M. J., Riedemann, S., Vega, I., Ibarra, H., Ercilla, G., and Haro, I., “Use of linear and multiple antigenic peptides in the immunodiagnosis of acute hepatitis: A virus infection,” J. Immunol. Methods 234, 23-34, 2000.
Gribskov, M. et al., eds., Sequence Analysis Primer, M Stockton Press, New York, 1991.
Griffin, A. M., et al, eds., Computer Analysis of Sequence Data, Part I, Humana Press, New Jersey, 1994.
Gulbis, B. et al., Hum Pathol 24(12):1271-1285, 1993.
Hardy, M. E., “Norovirus protein structure and function,” FEMS Microbiol Lett 253, 1-8, 2005.
Hardy, M. E., Tanaka, T. N., Kitamoto, N., White, L. J., Ball, J. M., Jiang, X., and Estes, M. K., “Antigenic mapping of the recombinant Norwalk virus capsid protein using monoclonal antibodies,” Virology 217, 252-261, 1996.
Hoess, R. H., “Protein design and phage display,” Chem Rev 101, 3205-3218, 2001.
Hong, S., Leroueil, P. R., Majoros, I. J., Orr, B. G., Baker, J. R., Jr., and Banaszak Holl, M. M., “The binding avidity of a nanoparticle-based multivalent targeted drug delivery platform,” Chem Biol 14, 107-115, 2007.
Hutson, A. M., Atmar, R. L., and Estes, M. K., “Norovirus disease: changing epidemiology and host susceptibility factors,” Trends Microbiol 12, 279-287, 2004.
ICTBdB Management (2006). 00.012.0.03. Norovirus. In: ICTVdB—The Universal Virus Database, version 4. Buchen-Osmond, C. (Ed), Columbioa University, New York, USA Kaplitt et al., Nat'l Genet., 8:148-153, 1994.
Kehoe, J. W., and Kay, B. K., “Filamentous phage display in the new millennium,” Chem Rev 105, 4056-4072, 2005.
Kitamoto, N., Tanaka, T., Natori, K., Takeda, N., Nakata, S., Jiang, X., and Estes, M. K., “Cross-reactivity among several recombinant calicivirus virus-like particles (VLPs) with monoclonal antibodies obtained from mice immunized orally with one type of VLP,” J Clin Microbiol 40, 2459-2465, 2002.
Kyte, J., Doolittle, R. F., “A simple method for displaying the hydropathic character of a protein,” J. Mol. Biol., May 5; 157(1):105-32, 1982.
Lee, C. C., MacKay, J. A., Frechet, J. M. J., and Szoka, F. C., “Designing dendrimers for biological applications,” Nature Biotechnology 23, 1517-1526, 2005.
Lee, R. T., and Lee, Y. C., “Affinity enhancement by multivalent lectin-carbohydrate interaction,” Glycoconj J 17, 543-551, 2000.
Lesk, A. M., ed., Computational Molecular Biology, Oxford University Press, New York, 1988.
Mammen, M., Choi, S., and Whitesides, G. M., “Polyvalent interactions in biological systems: implications for design and use of multivalent ligands and inhibitors,” Angew. Chem. Int. Ed. 37, 2754-2794, 1998.
Mammen, M., Dahmann, G., and Whitesides, G. M., “Effective inhibitors of hemagglutination by influenza virus synthesized from polymers having active ester groups: Insight into mechanism of inhibition,” J Med Chem 38, 4179-4190, 1995.
Mann et al., Cell, 33:153-159, 1983.
Merrifield, Science, 232: 341-347, 1986.
Nakamura et al., In: Handbook of Experimental Immunology (4th Ed.), Weir, E., Herzenberg, L. A., Blackwell, C., Herzenberg, L. (eds), Vol. 1, Chapter 27, Blackwell Scientific Publ., Oxford, 1987.
Nicolas and Rubenstein, In: Vectors: A survey of molecular cloning vectors and their uses, Rodriguez and Denhardt (eds.), Stoneham: Butterworth, pp. 494-513, 1988.
Niederhafner, P., Sebestik, J., and Jezek, J., “Peptide dendrimers,” J Pept Sci 11, 757-788, 2005.
Noren, K. A., and Noren, C. J., “Construction of high-complexity combinatorial phage display peptide libraries,” Methods 23, 169-178, 2001.
Parker, T. D., Kitamoto, N., Tanaka, T., Hutson, A. M., and Estes, M. K., “Identification of Genogroup I and Genogroup II broadly reactive epitopes on the norovirus capsid,” J Virol 79, 7402-7409, 2005.
Petrenko, V. A., and Vodyanoy, V. J., “Phage display for detection of biological threat agents,” J Microbiol Methods 53, 253-262, 2003.
Prasad, B. V., Hardy, M. E., Dokland, T., Bella, J., Rossmann, M. G., and Estes, M. K., “X-ray crystallographic structure of the Norwalk virus capsid,” Science 286, 287-290, 1999.
Radford, A. D., Gaskell, R. M., and Hart, C. A., “Human norovirus infection and the lessons from animal caliciviruses,” Curr Opin Infect Dis 17, 471-478, 2004.
Ridgeway, In: Rodriguez R. L., Denhardt D. T., ed., “Vectors: A survey of molecular cloning vectors and their uses,” Stoneham: Butterworth, pp. 467-492, 1988.
Samulski et al., J. Virol., 61(10):3096-3101, 1987.
Sidhu, S. S., “Phage display in pharmaceutical biotechnology,” Curr Opin Biotechnol 11, 610-616, 2000.
Smith, D. W., ed., Biocomputing: Informatics and Genome Projects, Academic Press, New York, 1993.
Smith, G. P., and Petrenko, V. A., “Phage Display,” Chem Rev 97, 391-410, 1997.
Stratford-Perricaudet L. D., Briand P., Perricaudet M., “Feasibility of adenovirus-mediated gene transfer in vivo,” Bone Marrow Transplant; 9 Suppl 1:151-2, 1992.
Stewart and Young, Solid Phase Peptide Synthesis, 2d. ed., Pierce Chemical Co., 1984.
Tam et al., J. Am. Chem. Soc., 105:6442, 1983.
Tan, M., and Jiang, X., “Norovirus and its histo-blood group antigen receptors: an answer to a historical puzzle,” Trends Microbiol 13, 285-293, 2005.
Temin, In: Gene Transfer, Kucherlapati (ed.), New York: Plenum Press, pp. 149-188, 1986.
von Heinje, G., Sequence Analysis in Molecular Biology, Academic Press, 1987.
Wu and Wu, Biochemistry, 27:887-892, 1988.
Yoda, T et al., J. Clin. Microbiol., 41(6):2367-71, 2003.

1. A composition comprising a peptide, wherein the peptide binds to a Norovirus or a Norovirus-like particle.

2. The composition of claim 1, wherein the peptide is provided on a solid support structure.

3. The composition of claim 1, wherein the Norovirus is a Norwalk virus.

4. The composition of claim 1, wherein the peptide is 3-50 amino acids in length, 3-20 amino acids in length, 10-20 amino acids in length, or 12 amino acids in length.

5. (canceled)

6. (canceled)

7. (canceled)

8. The composition of claim 2, wherein multiple copies of the peptide are provided on the solid support structure.

9. The composition of claim 8, where from 2 and 50 copies of the peptide are provided on the solid support structure.

10. The composition of claim 2, wherein the peptide and solid support structure are comprised in a dendrimer.

11. The composition of claim 2, wherein the solid support structure is selected from the group consisting of a membrane, a filter, a chip, a slide, a wafer, a fiber, a magnetic or nonmagnetic bead, a gel, tubing, a strip, a plate, a rod, a polymer, a particle, a microparticle, a capillary, a column, a resin, a protein and a combination thereof.

12. The composition of claim 11, wherein the solid support structure comprises a resin.

13. The composition of claim 12, wherein the resin is an Fmoc MAP resin.

14. The composition of claim 13, wherein the resin is an Fmoc-8-branch MAP resin.

15. The composition of claim 11, wherein the solid support structure comprises a protein.

16. The composition of claim 15, wherein the protein is bovine serum albumin (BSA).

17. The composition of claim 10, wherein the dendrimer is a monovalent dendrimer, a multivalent dendrimer, a divalent dendrimer, a trivalent dendrimer, a tetravalent dendrimer, a pentavalent dendrimer, a hexavalent dendrimer, a heptavalent dendrimer, an octovalent dendrimer, a nanovalent dendrimer, or a decavalent denrimer.

18. (canceled)

19. (canceled)

20. (canceled)

21. (canceled)

22. (canceled)

23. (canceled)

24. (canceled)

25. (canceled)

26. (canceled)

27. (canceled)

28. The composition of claim 10, wherein the dendrimer has more branches than a decavalent dendrimer.

29. The composition of claim 28, wherein the dendrimer has 11-30 branches, 11-20 branches, 12 branches, or 16 branches.

30. (canceled)

31. (canceled)

32. (canceled)

33. The composition of claim 1, wherein the C-terminus of the peptide comprises Pro-Xaa-Xaa.

34. The composition of claim 33, wherein the peptide is less than 50 amino acids in length, less than 15 amino acids in length, or is 12 amino acids in length.

35. (canceled)

36. (canceled)

37. The composition of claim 1, wherein the peptide comprises SEQ ID NO: 4.

38. The composition of claim 1, wherein the peptide is SEQ ID NO: 1 SEQ ID NO: 2, SEQ ID NO: 8, SEQ ID NO: 10, SEQ ID NO: 11, SEQ ID NO: 3, or mixtures thereof.

39. The composition of claim 1, wherein the peptide is SEQ ID NO: 2.

40. The composition of claim 1, wherein the peptide binds to a Norovirus genogroup selected from the group consisting of genogroup I, genogroup II, genogroup III, genogroup IV, genogroup V and a combination thereof.

41. The composition of claim 40, wherein the peptide binds to genogroup I or genogroup II.

42. (canceled)

43. (canceled)

44. The composition of claim 42, wherein the peptide binds to the Norwalk virus.

45. The composition of claim 43, wherein the peptide binds a strain of the genogroup II Norovirus selected from the group consisting of Houston, Snow Mountain, Grimsby and a combination thereof.

46. The composition of claim 1, wherein the peptide binds to the P domain of said Norovirus or Norovirus-like particle.

47. A method of detecting a Norovirus or a Norovirus-like particle in a sample comprising the steps of:
exposing the sample to a peptide that binds to a Norovirus or a Norovirus-like particle; and
detecting binding of said Norovirus or Norovirus-like particle to the peptide, wherein binding indicates the presence of a Norovirus or Norovirus-like particle in the sample.

48. The method of claim 47, wherein the peptide is provided on a solid support structure.

49. The method of claim 47, wherein the detecting step comprises
obtaining the Norovirus or Norovirus-like particle in a complex with said peptide to produce a Norovirus or Norovirus-like particle and peptide complex; and
providing an antibody that immunologically reacts with the complex, wherein when said antibody immunologically reacts with the complex said Norovirus or Norovirus-like particle is detected.

50. The method of claim 49, wherein the providing step comprises detection by a secondary antibody.

51. The method of claim 49, wherein the providing step comprises detection by a tertiary antibody.

52. The method of claim 49, wherein the detection comprises detection of a label, wherein the label is selected from the group consisting of a florescent label, a colorometric label, and a radioactive label.

53. (canceled)

54. (canceled)

55. (canceled)

56. (canceled)

57. (canceled)

58. The method of claim 48, wherein multiple copies of the peptide are provided on the solid support structure.

59. The method of claim 58, where from 2 and 50 copies of the peptide are provided on the solid support structure.

60. The method of claim 48, wherein the peptide and solid support structure are comprised in a dendrimer.

61. The method of claim 48, wherein the solid support structure is selected from the group consisting of a membrane, a filter, a chip, a slide, a wafer, a fiber, a magnetic or nonmagnetic bead, a gel, tubing, a strip, a plate, a rod, a polymer, a particle, a microparticle, a capillary, a column, a resin, a protein and a combination thereof.

62. The method of claim 61, wherein the solid support structure comprises a resin.

63. The method of claim 62, wherein the resin is an Fmoc MAP resin.

64. The method of claim 63, wherein the resin is an Fmoc-8-branch MAP resin.

65. The method of claim 61, wherein the solid support structure comprises a protein.

66.-235. (canceled)
